(19) United States
(12) Patent Application Publication
(10) Pub. No.: US 2010/0105814 A1
(43) Pub. Date: Apr. 29, 2010

(54) ULTRAVIOLET ABSORBENT AND POLYMER MATERIAL CONTAINING THE SAME

(75) Inventors: Akihiro KANEKO, Ashigarakami-gun (JP); Toshihiko Yawata, Minami-ashigara-shi (JP); Yoshihiko Fujie, Haibara-gun (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 12/606,638

(22) Filed: Oct. 27, 2009

(30) Foreign Application Priority Data
Oct. 27, 2008 (JP) 2008-276103

(51) Int. Cl. C08K 5/47; C08K 5/45; C07D 417/04; C07D 413/04

(52) U.S. Cl. 524/83; 524/84; 548/180; 548/243

(57) ABSTRACT

An ultraviolet absorbent, containing a compound represented by formula (1):

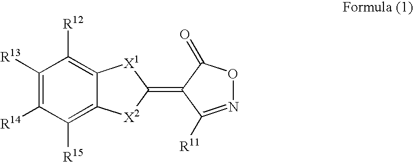

wherein X¹ and X² each are an oxygen atom, a sulfur atom, or —NR¹⁶—; R¹¹, R¹², R¹³, R¹⁴, R¹⁵ and R¹⁶ each are a hydrogen atom or a monovalent substituent.

### FIELD OF THE INVENTION

The present invention relates to an ultraviolet absorbent and a polymer material containing the ultraviolet absorbent.

### BACKGROUND OF THE INVENTION

Ultraviolet absorbents have been used in combination with various resins for providing the resins with ultraviolet-absorptivity. Both inorganic and organic ultraviolet absorbents are used. The inorganic ultraviolet absorbents (see, for example, JP-A-5-339033 (“JP-A” means unexamined published Japanese patent application), JP-A-5-345639 and JP-A-6-56466) are superior in durability properties such as weather resistance and heat resistance. However, the freedom in selecting the compound is limited, because the absorption wavelength is determined by the band gap of the compound. In addition, there is no inorganic absorbent that absorbs the light in a long-wavelength ultraviolet (UV-A) range of 320 to 400 nm And any such absorbent that absorbs long-wavelength ultraviolet would have color because it would have absorption also in the visible range.

In contrast, the freedom in designing the absorbent structure is much wider for organic ultraviolet absorbents, and thus, it is possible to obtain absorbents having various absorption wavelengths by designing the absorbent chemical structure properly.

Various organic ultraviolet absorbent systems have been studied, and for absorption in the long-wavelength ultraviolet range, it is conceivable either to use an absorbent having the wavelength of maximal absorption in the long-wavelength ultraviolet range or to use a high concentration of absorbent. However, the absorbents described in, for example, JP-A-6-145387 and JP-A-2003-177235 having the wavelength of maximal absorption in the long-wavelength ultraviolet range are inferior in light stability, and their absorption capacity declines over time.

In contrast, benzophenone- and benzotriazole-based ultraviolet absorbents are relatively superior in light stability, and increase in concentration or film thickness leads to relatively clean blocking of the light in the longer-wavelength range (see, for example, JP-T-2005-517787 (“JP-T” means published Japanese translation of PCT application) and JP-A-7-285927). However, when such an ultraviolet absorbent is applied as mixed with a resin or the like, the film thickness is limited to several tens of μm at the most. For utilizing the film thickness to block the light in the longer-wavelength range, it is necessary to add the ultraviolet absorbent to a considerably high concentration. In such a case, there were problems of precipitation of the ultraviolet absorbent and bleed-out during long-term use. In addition, an ultraviolet absorbent having the wavelength of maximal absorption in the long-wavelength ultraviolet range but also having absorption in the range of 400 nm or more becomes yellowish when used, only to deteriorate the tone of the color image after transmission. Accordingly, increase in concentration leads to distinct problems. Under the circumstances, there is a need for an ultraviolet absorbent that blocks the light in a wide ultraviolet range and yet has no absorption in the visible range. In addition, among benzophenone-based and benzotriazole-based ultraviolet absorbents, there are some ultraviolet absorbents that may cause concern about skin irritation and accumulation in body.

### SUMMARY OF THE INVENTION

The present invention resides in an ultraviolet absorbent, comprising a compound represented by formula (1):

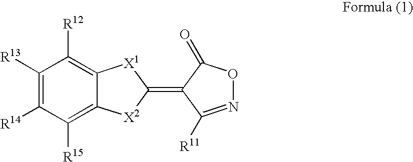

wherein X¹ and X² each independently represent an oxygen atom, a sulfur atom, or —NR¹⁶—; R¹¹, R¹², R¹³, R¹⁴, R¹⁵ and R¹⁶ each independently represent a hydrogen atom or a monovalent substituent.

Further, the present invention resides in a polymer material, comprising the ultraviolet absorbent described above, and at least one kind of polymer substance.

Further, the present invention resides in a compound represented by formula (2).

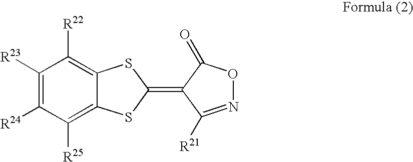

wherein R²¹ represents a substituted or unsubstituted alkyl group; and R²², R²³, R²⁴ and R²⁵ each independently represent a hydrogen atom or a monovalent substituent.

Other and further features and advantages of the invention will appear more fully from the following description.

### DETAILED DESCRIPTION OF THE INVENTION

Focusing on the isooxazolone skeleton as described in, for example, European Patent No. 412379, the present inventors have repeated detailed syntheses and evaluations of various isooxazolone compounds. As a result, the present inventors have found that compounds represented by the below-described formula (1) or (2) having a 1,3-benzodithiolan skeleton or another azole skeleton (for example, benzothiazole) or the above-described isooxazolone skeleton in the molecule shows a characteristic waveform having an excellent absorption capacity of, in particular, long-wavelength ultraviolet. Namely, the waveform is a spectral form wherein there is no absorption in a visible range of the long-wavelength side, whereas a sufficient absorption in an ultraviolet range of the long-wavelength side. In other word, the spectral form is precipitous at the long-wave side.

In view of production of a less colored 410 nm cut filter (the cut filter means a polymer film having 1% or less of transmittance in the specified wavelength) that is difficult to be produced using a general-purposed ultraviolet absorbent as described below in Example, it was unpredictable that the ultraviolet absorbent of the present invention has a favorable absorption form.

Further, the present inventors have found that the compound represented by the below-described formula (1) or (2) is also excellent both in light fastness and solubility. The inventors have found that it was possible, by using the compound having a particular structure higher in light fastness in a polymer material, to give a polymer material resistant to precipitation of the compound or bleeding out during long-term use, superior in long-wavelength ultraviolet absorption capacity, and superior in lightfastness while keeping the absorption capacity for an extended period of time. The present invention was made based on these findings.

According to the present invention, there are provided the following means:

[1] An ultraviolet absorbent, comprising a compound represented by formula (1):

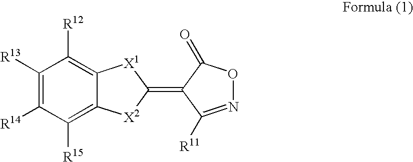

wherein X¹ and X² each independently represent an oxygen atom, a sulfur atom, or —NR¹⁶—; R¹¹, R¹², R¹³, R¹⁴, R¹⁵ and R¹⁶ each independently represent a hydrogen atom or a monovalent substituent.

[2] The ultraviolet absorbent described in the above item [1], wherein, in formula (1), X¹ and X² each are a sulfur atom.
[3] A polymer material, comprising: the ultraviolet absorbent described in the above item [1] or [2], and at least one kind of polymer substance.
[4] The polymer material described in the above item [3], wherein the polymer substance is at least one kind of substance selected from the group consisting of acrylic acid-based polymers, polyester-based polymers and polycarbonate-based polymers.
[5] The polymer material described in the above item [3] or [4], wherein a glass transition point (Tg) of the polymer substance is −80° C. or higher and 200° C. or lower.
[6] The polymer material described in any one of the above items [3] to [5], wherein the polymer substance is a polyacrylate ester, a polycarbonate or a polyethylene terephthalate.
[7] The polymer material described in any one of the above items [3] to [6], wherein the polymer substance is the polyethylene terephthalate; and wherein the ultraviolet absorbent is contained in an amount of 0.1 mass % to 50 mass % with respect to 100 mass % of the polyethylene terephthalate.
[8] The polymer material described in the above item [7], wherein the polymer material is a polymer material prepared by melt-kneading of the polyethylene terephthalate and the ultraviolet absorbent at a temperature of 200° C. or higher.
[9] The polymer material described in any one of the above items [3] to [6], wherein the polymer substance is the polyacrylate ester or the polycarbonate; and wherein the ultraviolet absorbent is contained in an amount of 0.1 mass % to 50 mass % with respect to 100 mass % of the polyacrylate ester or polycarbonate.
[10] The polymer material described in the above item [9], wherein the polymer material is a polymer material prepared by dissolving the polyacrylate ester or the polycarbonate and the ultraviolet absorbent in a solvent having a boiling point of 200° C. or lower to give a solution, and applying the solution on a base substrate.
[11] A compound represented by formula (2):

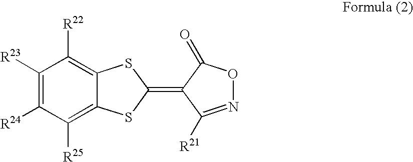

wherein R²¹ represents a substituted or unsubstituted alkyl group; and R²², R²³, R²⁴ and R²⁵ each independently represent a hydrogen atom or a monovalent substituent.

The present invention is described below in detail. The constitutional requirements described below may be embodied on the basis of the representative embodiments of the present invention. However the present invention is not limited to such embodiments. In the present specification, “to” denotes a range including numerical values described before and after it as a minimum value and a maximum value.

### [Compound Represented by Formula (1)]

The ultraviolet absorbent (long-wavelength ultraviolet absorbent) comprising the compound represented by Formula (1) will be described below.

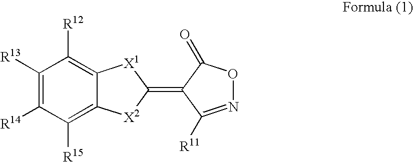

(In formula (1), X¹ and X² each independently represent an oxygen atom, a sulfur atom, or —NR¹⁶—; R¹¹, R¹², R¹³, R¹⁴, R¹⁵ and R¹⁶ each independently represent a hydrogen atom or a monovalent substituent.)

The compound represented by formula (1) is a merocyanine-based dye having an isooxazolone skeleton as an acidic heterocyclic ring. (The acidic hetero ring as referred to herein is defined, for example, by James, “The Theory of the Photographic Process”, 4th Ed., MacMillan Publishing, 1977, p. 197. The merocyanine-based dye as referred to herein is defined, for example, by S. Ohkawara, K. Matsuoka, T. Hirashima & T. Kitao, “Functional Dye”, Kodansha Scientific, 1992, p. 79.)

The compounds themselves represented by formula (1) are known as sensitizing dye for light-sensitive compositions that are used, for example, for printing materials or the like (see, for example, JP-A-3-54566). However, utility of the compound as an ultraviolet absorbent has not been reported by anyone yet. Thus, it is unexpected that the compound represented by formula (1) has an especially excellent property as a long-wavelength ultraviolet absorbing material.

In formula (1), X¹ and X² each independently represent an oxygen atom, sulfur atom, or —NR¹⁶—; R¹⁶ represents a hydrogen atom or a monovalent substituent.

Preferably, X¹ and X² each independently represent a sulfur atom or —NR¹⁶—. Particularly preferably, both of X¹ and X² represent a sulfur atom.

R¹⁶ preferably represents a substituted or unsubstituted alkyl group having 1 to 30 carbon atoms or a substituted or unsubstituted aryl group having 6 to 10 carbon atoms, more preferably a substituted or unsubstituted alkyl group having 1 to 20 carbon atoms, particularly preferably a substituted or unsubstituted alkyl group having 1 to 18 carbon atoms.

In formula (1), R¹², R¹³, R¹⁴, R¹⁵ and R¹⁶ each independently represent a hydrogen atom or a monovalent substituent. The monovalent substituent is not particularly limited. Examples thereof include a halogen atom, an aliphatic group [a saturated aliphatic group (this term includes an alkyl group, and a cyclic saturated aliphatic group including a cycloalkyl group, a bicycloalkyl group, a crosslinked cyclic saturated hydrocarbon group, and a spiro-saturated hydrocarbon group), an unsaturated aliphatic group (this term includes a linear unsaturated aliphatic group having a double bond or a triple bond, such as an alkenyl group, an alkynyl group; and a cyclic unsaturated aliphatic group including a cycloalkenyl group, a bicycloalkenyl group, a crosslinked cyclic unsaturated hydrocarbon group, and a spiro-unsaturated hydrocarbon group)], an aryl group (preferably a substituted or unsubstituted phenyl group), a heterocyclic group (preferably a 5- to 8-membered, alicyclic, aromatic or heterocyclic ring having an oxygen atom, a sulfur atom or a nitrogen atom as the ring-constitutive atom, and it may be condensed with a ring such as an aliphatic ring, an aromatic ring and a heterocyclic ring), a cyano group, an aliphatic oxy group (typically an alkoxy group), an aryloxy group, an acyloxy group, a carbamoyloxy group, an aliphatic oxycarbonyloxy group (typically an alkoxycarbonyloxy group), an aryloxycarbonyloxy group, an amino group [including an aliphatic amino group (typically an alkylamino group), an anilino group, and a heterocyclic amino group], an acylamino group, an aminocarbonylamino group, an aliphatic oxycarbonylamino group (typically an alkoxycarbonylamino group), an aryloxycarbonylamino group, a sulfamoylamino group, an aliphatic (typically an alkyl) or aryl sulfonylamino group, an aliphatic thio group (typically an alkylthio group), an arylthio group, a sulfamoyl group, an aliphatic (typically an alkyl) or aryl-sulfinyl group, an aliphatic (typically an alkyl) or aryl-sulfonyl group, an acyl group, an aryloxycarbonyl group, an aliphatic oxycarbonyl group (typically an alkoxycarbonyl group), a carbamoyl group, an aryl or heterocyclic azo group, an imide group, an aliphatic oxysulfonyl group (typically an alkoxysulfonyl group), an aryloxysulfonyl group, a hydroxyl group, a nitro group, a carboxyl group, and a sulfo group. These groups may be further substituted with a substituent (for example, with the substituent mentioned in the above).

The substituents of R¹², R¹³, R¹⁴, R¹⁵ and R¹⁶, and substituents which may substitute to each substituent of R¹², R¹³, R¹⁴, R¹⁵ and R¹⁶, are described in greater details blow.

The halogen atom of and on R¹², R¹³, R¹⁴, R¹⁵ and R¹⁶ includes a fluorine atom, a chlorine atom, a bromine atom, and an iodine atom. Of these, a chlorine atom and a bromine atom are preferable, a chlorine atom is particularly preferable.

The aliphatic group of and on R¹², R¹³, R¹⁴, R¹⁵ and R¹⁶ includes a linear, branched and cyclic aliphatic groups. The term “saturated aliphatic group” includes an alkyl group, a cycloalkyl group, and a bicycloalkyl group; and these groups may have a substituent. The carbon numbers of these substituents is preferably from 1 to 30. Examples of the alkyl group include a methyl group, an ethyl group, an n-propyl group, an isopropyl group, a t-butyl group, an n-octyl group, an eicosyl group, a 2-chloroethyl group, a 2-cyanoethyl group, a benzyl group and a 2-ethylhexyl group. The cycloalkyl group includes a substituted or unsubstituted cycloalkyl group. The substituted or unsubstituted cycloalkyl group is preferably a cycloalkyl group having 3 to 30 carbon atoms. Examples of the cycloalkyl group include a cyclohexyl group, a cyclopentyl group and a 4-n-dodecylcyclohexyl group. The bicycloalkyl group is preferably a substituted or unsubstituted bicycloalkyl group having 5 to 30 carbon atoms, i.e., a monovalent group obtained by removing one hydrogen atom from a bicycloalkane having 5 to 30 carbon atoms. Examples of the bicycloalkyl group include a bicyclo[1,2,2]heptan-2-yl group and a bicyclo[2,2,2]octan-3-yl group, and a tricyclo or higher structure having three or more ring structures.

The unsaturated aliphatic group of and on R¹², R¹³, R¹⁴, R¹⁵ and R¹⁶ includes a linear, branched, and cyclic unsaturated aliphatic groups. The unsaturated aliphatic group includes an alkenyl group, a cycloalkenyl group, a bicycloalkenyl group and an alkynyl group. The alkenyl group includes a linear, branched, and cyclic substituted or unsubstituted alkenyl groups. The alkenyl group is preferably a substituted or unsubstituted alkenyl group having 2 to 30 carbon atoms. Examples of the alkenyl group include a vinyl group, an allyl group, a prenyl group, a geranyl group, and an oleyl group. The cycloalkenyl group is preferably a substituted or unsubstituted cycloalkenyl group having 3 to 30 carbon atoms, i.e., a monovalent group obtained by removing one hydrogen atom from a cycloalkene having 3 to 30 carbon atoms.

Examples of the cycloalkenyl group include a 2-cyclopenten-1-yl group and a 2-cyclohexen-1-yl group. The bicycloalkenyl group includes a substituted or unsubstituted bicycloalkenyl group, and preferably a substituted or unsubstituted bicycloalkenyl group having 5 to 30 carbon atoms, i.e., a monovalent group obtained by removing one hydrogen atom from a bicycloalkene having one double bond. Examples of the bicycloalkenyl group include a bicyclo[2,2,1]hept-2-en-1-yl group and a bicyclo[2,2,2]oct-2-en-4-yl group. The alkynyl group is preferably a substituted or unsubstituted alkynyl group having 2 to 30 carbon atoms, e.g., an ethynyl group, or a propargyl group.

The aryl group of or on R¹², R¹³, R¹⁴, R¹⁵ and R¹⁶ is preferably a substituted or unsubstituted aryl group having 6 to 30 carbon atoms, e.g., a phenyl group, a p-tolyl group, a naphthyl group, an m-chlorophenyl group, or an o-hexadecanoylaminophenyl group. The aryl group is more preferably a phenyl group which may have a substituent.

The heterocyclic group of or on R¹², R¹³, R¹⁴, R¹⁵ and R¹⁶ is a monovalent group obtained by removing one hydrogen atom from a substituted or unsubstituted, aromatic or non-aromatic heterocyclic compound, which may be condensed to another ring. The heterocyclic group is preferably a 5- or 6-membered heterocyclic group. The hetero atom(s) constituting the heterocyclic group is preferably an oxygen atom, a sulfur atom, or a nitrogen atom. The heterocyclic group is more preferably a 5- or 6-membered aromatic heterocyclic group having 3 to 30 carbon atoms. The hetero ring in the heterocyclic group are exemplified below: a pyridine ring, a pyrazine ring, a pyridazine ring, a pyrimidine ring, a triazine ring, a quinoline ring, an isoquinoline ring, a quinazoline ring, a cinnoline ring, a phthalazine ring, a quinoxaline ring, a pyrrole ring, an indole ring, a furan ring, a benzofuran ring, a thiophene ring, a benzothiophene ring, a pyrazole ring, an imidazole ring, a benzimidazole ring, a triazole ring, an oxazole ring, a benzoxazole ring, a thiazole ring, a benzothiazole ring, an isothiazole ring, a benzisothiazole ring, a thiadiazole ring, an isoxazole ring, a benzisoxazole ring, a pyrrolidine ring, a piperidine ring, a piperazine ring, an imidazolidine ring and a thiazoline ring.

The aliphatic oxy group (as a representative example, an alkoxy group) of or on R¹², R¹³, R¹⁴, R¹⁵ and R¹⁶ includes a substituted or unsubstituted aliphatic oxy group (as a representative example, alkoxy group). The substituted or unsubstituted aliphatic oxy group preferably has 1 to 30 carbon atoms, e.g., a methoxy group, an ethoxy group, an isopropoxy group, an n-octyloxy group, a methoxyethoxy group, a hydroxyethoxy group, or a 3-carboxypropoxy group.

The aryloxy group of or on R¹², R¹³, R¹⁴, R¹⁵ and R¹⁶ is preferably a substituted or unsubstituted aryloxy group having 6 to 30 carbon atoms, e.g., a phenoxy group, a 2-methylphenoxy group, a 4-t-butylphenoxy group, a 3-nitrophenoxy group, or a 2-tetradecanoylaminophenoxy group. The aryloxy group is more preferably a phenoxy group which may have a substituent.

The acyloxy group of or on R¹², R¹³, R¹⁴, R¹⁵ and R¹⁶ is preferably a formyloxy group, a substituted or unsubstituted alkylcarbonyloxy group having 2 to 30 carbon atoms, or a substituted or unsubstituted arylcarbonyloxy group having 6 to 30 carbon atoms, e.g., a formyloxy group, an acetyloxy group, a pivaloyloxy group, a stearoyloxy group, a benzoyloxy group, or a p-methoxyphenylcarbonyloxy group.

The carbamoyloxy group of or on R¹², R¹³, R¹⁴, R¹⁵ and R¹⁶ is preferably a substituted or unsubstituted carbamoyloxy group having 1 to 30 carbon atoms, e.g., an N,N-dimethylcarbamoyloxy group, an N,N-diethylcarbamoyloxy group, a morpholinocarbonyloxy group, an N,N-di-n-octylaminocarbonyloxy group, or an N-n-octylcarbamoyloxy group.

The aliphatic oxy carbonyloxy group (as a representative example, an alkoxycarbonyloxy group) of or on R¹², R¹³, R¹⁴, R¹⁵ and R¹⁶ is preferably an aliphatic oxy carbonyloxy group having 2 to 30 carbon atoms. The aliphatic oxy carbonyloxy group may have a substituent. There can be exemplified a methoxycarbonyloxy group, an ethoxycarbonyloxy group, a t-butoxycarbonyloxy group, or an n-octylcarbonyloxy group.

The aryloxycarbonyloxy group of or on R¹², R¹³, R¹⁴, R¹⁵ and R¹⁶ is preferably a substituted or unsubstituted aryloxycarbonyloxy group having 7 to 30 carbon atoms, e.g., a phenoxycarbonyloxy group, a p-methoxyphenoxycarbonyloxy group, or a p-n-hexadecyloxyphenoxycarbonyloxy group. The aryloxycarbonyloxy group is more preferably a phenoxycarbonyloxy group which may have a substituent.

The amino group of or on R¹², R¹³, R¹⁴, R¹⁵ and R¹⁶ includes an unsubstituted amino group, an aliphatic amino group (as a representative example, an alkylamino group), an arylamino group, and a heterocyclic amino group. The amino group is preferably a substituted or unsubstituted aliphatic amino group (as a representative example, alkylamino group) having 1 to 30 carbon atoms, or a substituted or unsubstituted arylamino group having 6 to 30 carbon atoms, e.g., an amino group, a methylamino group, a dimethylamino group, an anilino group, an N-methyl-anilino group, a diphenylamino group, a hydroxyethylamino group, a carboxyethylamino group, a sulfoethylamino group, a 3,5-dicarboxyanilino group, or a 4-quinolylamino group.

The acylamino group of or on R¹², R¹³, R¹⁴, R¹⁵ and R¹⁶ is preferably a formylamino group, a substituted or unsubstituted alkylcarbonylamino group having 1 to 30 carbon atoms, or a substituted or unsubstituted arylcarbonylamino group having 6 to 30 carbon atoms, e.g., a formylamino group, an acetylamino group, a pivaloylamino group, a lauroylamino group, a benzoylamino group, or a 3,4,5-tri-n-octyloxyphenylcarbonylamino group.

The aminocarbonylamino group of or on R¹², R¹³, R¹⁴, R¹⁵ and R¹⁶ is preferably a substituted or unsubstituted aminocarbonylamino group having 1 to 30 carbon atoms, e.g., a carbamoylamino group, an N,N-dimethylaminocarbonylamino group, an N,N-diethylaminocarbonylamino group, or a morpholinocarbonylamino group. In the aminocarbonylamino group, the term “amino” has the same meaning as “amino” in the above-described amino group.

The aliphatic oxy carbonylamino group (as a representative example, alkoxycarbonylamino group) of or on R¹², R¹³, R¹⁴, R¹⁵ and R¹⁶ is preferably a substituted or unsubstituted aliphatic oxy carbonylamino group having 2 to 30 carbon atoms, e.g., a methoxycarbonylamino group, an ethoxycarbonylamino group, a t-butoxycarbonylamino group, an n-octadecyloxycarbonylamino group, or an N-methyl-methoxycarbonylamino group.

The aryloxycarbonylamino group of or on R¹², R¹³, R¹⁴, R¹⁵ and R¹⁶ is preferably a substituted or unsubstituted aryloxycarbonylamino group having 7 to 30 carbon atoms, e.g., a phenoxycarbonylamino group, a p-chlorophenoxycarbonylamino group, or an m-(n-octyloxy)phenoxycarbonylamino group. The aryloxycarbonylamino group is more preferably a phenoxycarbonylamino group which may have a substituent.

The sulfamoylamino group of or on R¹², R¹³, R¹⁴, R¹⁵ and R¹⁶ is preferably a substituted or unsubstituted sulfamoylamino group having 0 to 30 carbon atoms, e.g., a sulfamoylamino group, an N,N-dimethylaminosulfonylamino group, or an N-n-octylaminosulfonylamino group.

The aliphatic- (as a representative example, alkyl-) or aryl-sulfonylamino group of or on R¹², R¹³, R¹⁴, R¹⁵ and R¹⁶ is preferably a substituted or unsubstituted aliphatic sulfonylamino group (as a representative example, alkylsulfonylamino group) having 1 to 30 carbon atoms, or a substituted or unsubstituted arylsulfonylamino group (preferably a phenylsulfonylamino group which may have a substituent(s)) having 6 to 30 carbon atoms, e.g., a methylsulfonylamino group, a butylsulfonylamino group, a phenylsulfonylamino group, a 2,3,5-trichlorophenylsulfonylamino group, or a p-methylphenylsulfonylamino group.

The aliphatic thio group (as a representative example, alkylthio group) of or on R¹², R¹³, R¹⁴, R¹⁵ and R¹⁶ is preferably a substituted or unsubstituted alkylthio group having 1 to 30 carbon atoms, e.g., a methylthio group, an ethylthio group, or an n-hexadecylthio group.

The aryl thio group of or on R¹², R¹³, R¹⁴, R¹⁵ and R¹⁶ is preferably a substituted or unsubstituted aryl thio group having 6 to 12 carbon atoms, e.g., a phenylthio group, a 1-naphthylthio group, or a 2-naphthylthio group. The sulfamoyl group of or on R¹², R¹³, R¹⁴, R¹⁵ and R¹⁶ is preferably a substituted or unsubstituted sulfamoyl group having 0 to 30 carbon atoms, e.g., an N-ethylsulfamoyl group, an N-(3-dodecyloxypropyl)sulfamoyl group, an N,N-dimethylsulfamoyl group, an N-acetylsulfamoyl group, an N-benzoylsulfamoly group, or an N—(N′-phenylcarbamoyl)sulfamoyl group.

The aliphatic- (as a representative example, alkyl-) or aryl-sulfinyl group of or on R¹², R¹³, R¹⁴, R¹⁵ and R¹⁶ is preferably a substituted or unsubstituted aliphatic sulfinyl group (as a representative example, alkylsulfinyl group) having 1 to 30 carbon atoms, or a substituted or unsubstituted arylsulfinyl group (preferably a phenylsulfinyl group which may have a substituent(s)) having 6 to 30 carbon atoms, e.g., a methylsulfinyl group, an ethylsulfinyl group, a phenylsulfinyl group, or a p-methylphenylsulfinyl group.

The aliphatic- (as a representative example, alkyl-) or aryl-sulfonyl group of or on R¹², R¹³, R¹⁴, R¹⁵ and R¹⁶ is preferably a substituted or unsubstituted aliphatic-sulfonyl group (as a representative example, alkylsulfonyl group) having 1 to 30 carbon atoms, or a substituted or unsubstituted arylsulfonyl group (preferably a phenylsulfonyl group which may have a substituent(s)) having 6 to 30 carbon atoms, e.g., a methylsulfonyl group, an ethylsulfonyl group, a phenylsulfonyl group, or a p-toluenesulfonyl group.

The acyl group of or on R¹², R¹³, R¹⁴, R¹⁵ and R¹⁶ is preferably a formyl group, a substituted or unsubstituted aliphatic carbonyl group (as a representative example, alkylcarbonyl group) having 2 to 30 carbon atoms, a substituted or unsubstituted arylcarbonyl group (preferably a phenylcarbonyl group which may have a substituent(s)) having 7 to 30 carbon atoms, or a substituted or unsubstituted heterocyclic carbonyl group having 4 to 30 carbon atoms and being bonded to said carbonyl group through a carbon atom, e.g., an acetyl group, a pivaloyl group, a 2-chloroacetyl group, a stearoyl group, a benzoyl group, a p-n-octyloxyphenylcarbonyl group, a 2-pyridylcarbonyl group, or a 2-furylcarbonyl group.

The aryloxycarbonyl group of or on R¹², R¹³, R¹⁴, R¹⁵ and R¹⁶ is preferably a substituted or unsubstituted aryloxycarbonyl group having 7 to 30 carbon atoms, e.g., a phenoxycarbonyl group, an o-chlorophenoxycarbonyl group, an m-nitrophenoxycarbonyl group, or a p-(t-butyl)phenoxycarbonyl group. The aryloxycarbonyl group is more preferably a phenoxycarbonyl group which may have a substituent.

The aliphatic oxycarbonyl group (as a representative example, alkoxycarbonyl group) of or on R¹², R¹³, R¹⁴, R¹⁵ and R¹⁶ is preferably a substituted or unsubstituted aliphatic oxycarbonyl group having 2 to 30 carbon atoms, e.g., a methoxycarbonyl group, an ethoxycarbonyl group, a t-butoxycarbonyl group, or an n-octadecyloxycarbonyl group.

The carbamoyl group of or on R¹², R¹³, R¹⁴, R¹⁵ and R¹⁶ is preferably a substituted or unsubstituted carbamoyl group having 1 to 30 carbon atoms, e.g., a carbamoyl group, an N-methylcarbamoyl group, an N,N-dimethylcarbamoyl group, an N,N-di-n-octylcarbamoyl group, or an N-(methylsulfonyl)carbamoyl group.

Examples of the aryl- or heterocyclic azo group that R¹², R¹³, R¹⁴, R¹⁵ and R¹⁶ may be or have include a phenylazo group, a 4-methoxyphenylazo group, a 4-pivaloylaminophenylazo group, and a 2-hydroxy-4-propanoylphenylazo group.

Examples of the imido group that R¹², R¹³, R¹⁴, R¹⁵ and R¹⁶ may be or have include an N-succinimido group and an N-phthalimido group.

The aliphatic oxysulfonyl group of or on R¹², R¹³, R¹⁴R¹⁵ and R¹⁶ (as a representative example, alkoxysulfonyl group) is preferably an aliphatic oxysulfonyl group having 1 to 30 carbon atoms, and may have a substituent group, e.g., a methoxysulfonyl group, an ethoxysulfonyl group, and a n-butoxysulfonyl group.

The aryloxysulfonyl group of or on R¹², R¹³, R¹⁴, R¹⁵ and R¹⁶ is preferably a substituted or unsubstituted aryloxysulfonyl group having 6 to 12 carbon atoms, and may have a substituent group, e.g., a phenoxysulfonyl group and a 2-naphthoxyphenyl group.

In addition to these substituents, examples of the substituent of or on R¹², R¹³, R¹⁴, R¹⁵ and R¹⁶ include a hydroxyl group, a cyano group, a nitro group, a sulfo group, a carboxyl group, and the like.

These groups may each further have a substituent. Examples of the substituent include the above-mentioned substituents.

R¹² and R¹⁵ each independently preferably represent a hydrogen atom, a substituted or unsubstituted alkyl group having 1 to 30 carbon atoms, a substituted or unsubstituted alkoxy group having 1 to 30 carbon atoms, a substituted or unsubstituted aryloxy group having 6 to 30 carbon atoms, a substituted or unsubstituted acyloxy group having 2 to 30 carbon atoms, a substituted or unsubstituted carbamoyloxy group having 1 to 30 carbon atoms, a hydroxyl group, or a halogen atom; more preferably represent a hydrogen atom, a substituted or unsubstituted alkoxy group having 1 to 20 carbon atoms, a substituted or unsubstituted aryloxy group having 6 to 20 carbon atoms, a substituted or unsubstituted acyloxy group having 2 to 20 carbon atoms, or a substituted or unsubstituted carbamoyloxy group having 1 to 20 carbon atoms; and particularly preferably represent a substituted or unsubstituted acyloxy group having 2 to 18 carbon atoms, or a substituted or unsubstituted carbamoyloxy group having 1 to 18 carbon atoms.

R¹³ and R¹⁴ each independently preferably represent a hydrogen atom, a substituted or unsubstituted alkyl group having 1 to 30 carbon atoms, a substituted or unsubstituted aryl group having 6 to 30 carbon atoms, a halogen atom, or cyano group; more preferably a hydrogen atom, or a substituted or unsubstituted alkyl group having 1 to 20 carbon atoms, particularly preferably a hydrogen atom.

In formula (1), R¹¹ represents a monovalent substituent. Examples of the monovalent substituent include an aliphatic group [a saturated aliphatic group (this term includes an alkyl group, and a cyclic saturated aliphatic group including a cycloalkyl group, a bicycloalkyl group, a crosslinked cyclic saturated hydrocarbon group, and a spiro-saturated hydrocarbon group), an unsaturated aliphatic group (this term includes a linear unsaturated aliphatic group having a double bond or a triple bond, such as an alkenyl group, or an alkynyl group; and a cyclic unsaturated aliphatic group including a cycloalkenyl group, a bicycloalkenyl group, a crosslinked cyclic unsaturated hydrocarbon group, and a Spiro-unsaturated hydrocarbon group)], an aryl group (preferably a substituted or unsubstituted phenyl group), a heterocyclic group (preferably a 5- to 8-membered, alicyclic, aromatic or heterocyclic ring having an oxygen atom, a sulfur atom or a nitrogen atom as the ring-constitutive atom, and it may be condensed with a ring such as an aliphatic ring, an aromatic ring and a heterocyclic ring), an aliphatic oxy group (typically an alkoxy group), an aryloxy group, an amino group [including an aliphatic amino group (typically an alkylamino group), an anilino group, and a heterocyclic amino group], an acylamino group, an aliphatic oxycarbonylamino group (typically an alkoxycarbonylamino group), an aryloxycarbonylamino group, a sulfamoylamino group, an aliphatic- (typically an alkyl-) or aryl-sulfonylamino group, an acyl group, an aryloxycarbonyl group, an aliphatic oxycarbonyl group (typically an alkoxycarbonyl group), a carbamoyl group, a hydroxyl group, and a carboxyl group.

R¹¹ may further have a substituent. Examples of the substituent include those listed as the substituent of R¹², R¹³, R¹⁴, R¹⁵ and R¹⁶.

R¹¹ preferably represent a substituted or unsubstituted alkyl group having 1 to 30 carbon atoms or a substituted or unsubstituted phenyl group, more preferably a substituted or unsubstituted alkyl group having 1 to 20 carbon atoms, particularly preferably a branched alkyl group having 1 to 5 carbon atoms.

Regarding a preferable combination of various substituents (atoms) that a compound represented by formula (1) may have, the compound having at least one substituent being the above mentioned preferable substituent is preferable, and the compound having more substituents being the above mentioned preferable substituents is more preferable, and the compound having all of the substituents being the above mentioned preferable substituents is the most preferable.

Examples of a preferred combination of X¹, X², R¹¹, R¹², R¹³, R¹⁴ and R¹⁵ in the compound represented by formula (1) include combinations wherein X¹ is a sulfur atom; X² is a sulfur atom or —NR¹⁶— (in which R¹⁶ represents a substituted or unsubstituted alkyl group having 1 to 30 carbon atoms); R¹¹ is a substituted or unsubstituted alkyl group having 1 to 30 carbon atoms; R¹² is a substituted or unsubstituted alkoxy group having 1 to 30 carbon atoms, a substituted or unsubstituted carbamoyloxy group having 1 to 30 carbon atoms, or a substituted or unsubstituted acyloxy group having 2 to 30 carbon atoms; R¹³ is a hydrogen atom, or a substituted or unsubstituted alkyl group having 1 to 30 carbon atoms; R¹⁴ is a hydrogen atom; and R¹⁵ is a substituted or unsubstituted alkoxy group having 1 to 30 carbon atoms, a substituted or unsubstituted carbamoyloxy group having 1 to 30 carbon atoms, or a substituted or unsubstituted acyloxy group having 2 to 30 carbon atoms.

In more preferred combinations thereof, X¹ is a sulfur atom; X² is a sulfur atom; R¹¹ is a substituted or unsubstituted alkyl group having 1 to 20 carbon atoms; R¹² is a substituted or unsubstituted alkoxy group having 1 to 20 carbon atoms, a substituted or unsubstituted carbamoyloxy group having 1 to 20 carbon atoms, or a substituted or unsubstituted acyloxy group having 2 to 20 carbon atoms; R¹³ is a hydrogen atom; R¹⁴ is a hydrogen atom; and R¹⁵ is a substituted or unsubstituted alkoxy group having 1 to 20 carbon atoms, a substituted or unsubstituted carbamoyloxy group having 1 to 30 carbon atoms, or a substituted or unsubstituted acyloxy group having 2 to 20 carbon atoms.

In the particularly preferred combinations thereof, X¹ is a sulfur atom; X² is a sulfur atom; R¹¹ is a branched alkyl group having 1 to 5 carbon atoms; R¹² is a substituted or unsubstituted acyloxy group having 2 to 18 carbon atoms, or a substituted or unsubstituted carbamoyloxy group having 1 to 18 carbon atoms; R¹³ is a hydrogen atom; R¹⁴ is a hydrogen atom; and R¹⁵ is a substituted or unsubstituted acyloxy group having 2 to 18 carbon atoms, or a substituted or unsubstituted carbamoyloxy group having 1 to 18 carbon atoms.

### [Compound Represented by Formula (2)]

A compound represented by formula (1) is preferably a compound represented by formula (2)

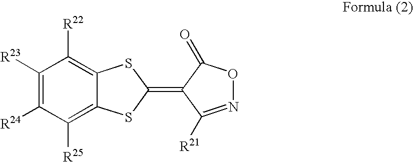

In formula (2), R²¹ represents a substituted or unsubstituted alkyl group; and R²², R²³, R²⁴ and R²⁵ each independently represent a hydrogen atom or a monovalent substituent.

In formula (2), R²¹ represents a substituted or unsubstituted alkyl group. The substituent that R²¹ may have, has the same meaning as the substituent on R¹¹, R¹², R¹³, R¹⁴, R¹⁵ and R¹⁶.

R²¹ preferably represent a substituted or unsubstituted alkyl group having 1 to 30 carbon atoms, more preferably a substituted or unsubstituted alkyl group having 1 to 20 carbon atoms, particularly preferably a branched alkyl group having 1 to 5 carbon atoms.

In formula (2), R²², R²³, R²⁴, and R²⁵ each have the same meaning as of R¹², R¹³, R¹⁴, and R¹⁵ in formula (1), and the preferable ranges are also the same.

Regarding a preferable combination of various substituents (atoms) that a compound represented by formula (2) may have, the compound having at least one substituent being the above mentioned preferable substituent is preferable, and the compound having more substituents being the above mentioned preferable substituents is more preferable, and the compound having all of the substituents being the above mentioned preferable substituents is the most preferable.

Examples of a preferred combination of R²¹, R²², R²³, R²⁴ in the compound represented by formula (2) include combinations wherein R²¹ is a substituted or unsubstituted alkyl group having 1 to 30 carbon atoms; R²² is a substituted or unsubstituted alkoxy group having 1 to 30 carbon atoms, a substituted or unsubstituted carbamoyloxy group having 1 to 30 carbon atoms, or a substituted or unsubstituted acyloxy group having 2 to 30 carbon atoms; R²³ is a hydrogen atom, or a substituted or unsubstituted alkyl group having 1 to 30 carbon atoms; R²⁴ is a hydrogen atom; and R²⁵ is a substituted or unsubstituted alkoxy group having 1 to 30 carbon atoms, a substituted or unsubstituted carbamoyloxy group having 1 to 30 carbon atoms, or a substituted or unsubstituted acyloxy group having 2 to 20 carbon atoms.

In more preferred combinations thereof, R²¹ is a substituted or unsubstituted alkyl group having 1 to 20 carbon atoms; R²² is a substituted or unsubstituted alkoxy group having 1 to 20 carbon atoms, a substituted or unsubstituted carbamoyloxy group having 1 to 20 carbon atoms, or a substituted or unsubstituted acyloxy group having 2 to 20 carbon atoms; R²³ is a hydrogen atom; R²⁴ is a hydrogen atom; and R²⁵ is a substituted or unsubstituted alkoxy group having 1 to 20 carbon atoms, a substituted or unsubstituted carbamoyloxy group having 1 to 30 carbon atoms, or a substituted or unsubstituted acyloxy group having 2 to 20 carbon atoms.

In the particularly preferred combinations thereof, R²¹ is a branched alkyl group having 1 to 5 carbon atoms; R²² is a substituted or unsubstituted acyloxy group having 2 to 18 carbon atoms, or a substituted or unsubstituted carbamoyloxy group having 1 to 18 carbon atoms; R²³ is a hydrogen atom; R²⁴ is a hydrogen atom; and R²⁵ is a substituted or unsubstituted acyloxy group having 2 to 18 carbon atoms, or a substituted or unsubstituted carbamoyloxy group having 1 to 18 carbon atoms.

A molecular weight of the compound represented by formula (1) is preferably 1500 or less, more preferably 1000 or less, and further more preferably from 280 to 900, from viewpoints of both ultraviolet absorption capacity and resistance to bleeding.

Hereinafter, specific examples of the compound represented by formula (1) are exemplified but the present invention is not limited thereto.

TABLE 1

| Compound No. | X¹ | X² | R¹¹ | R¹² | R¹³ | R¹⁴ | R¹⁵ | R¹⁶ |
|---|---|---|---|---|---|---|---|---|
| 1 | S | S | t-Bu | OCOCH(n-Bu)Et | H | H | OCOCH(n-Bu)Et | — |
| 2 | S | S | t-Bu | OCOCH(n-Bu)Et | Cl | H | OCOCH(n-Bu)Et | — |
| 3 | S | S | t-Bu | OCOCH(n-Bu)Et | t-Bu | H | OCOCH(n-Bu)Et | — |
| 4 | S | S | i-Pr | OCOCH(n-Bu)Et | t-Bu | H | OCOCH(n-Bu)Et | — |
| 5 | S | S | Me | OCOCH(n-Bu)Et | H | H | OCOCH(n-Bu)Et | — |
| 6 | S | S | Ph | OCOCH(n-Bu)Et | H | H | OCOCH(n-Bu)Et | — |
| 7 | S | S | t-Bu | OCOCH₃ | H | H | OCOCH₃ | — |
| 8 | S | S | t-Bu | OCOC(CH₃)₃ | OMe | H | OCOC(CH₃)₃ | — |
| 9 | S | S | t-Bu | O(n-Bu) | H | H | O(n-Bu) | — |
| 10 | S | S | t-Bu | O(n-Bu) | Br | H | O(n-Bu) | — |
| 11 | S | S | COOEt | OMe | H | H | OMe | — |
| 12 | S | S | Me | O(i-Pr) | H | H | O(i-Pr) | — |
| 13 | S | S | t-Bu | O(n-C₁₈H₃₇) | H | H | O(n-C₁₈H₃₇) | — |
| 14 | S | S | t-Bu | OCH₂CH(n-Bu)Et | H | H | OCH₂CH(n-Bu)Et | — |
| 15 | S | S | t-Bu | OCH₂COOEt | H | H | OCH₂COOEt | — |
| 16 | S | S | t-Bu | OCONEt₂ | H | H | OCONEt₂ | — |
| 17 | S | S | t-Bu | OCONEt₂ | Cl | H | OCONEt₂ | — |
| 18 | S | S | i-Pr | OCONEt₂ | H | H | OCONEt₂ | — |
| 19 | S | S | Me | OCONEt₂ | H | H | OCONEt₂ | — |
| 20 | S | S | Ph | OCONEt₂ | H | H | OCONEt₂ | — |
| 21 | S | S | Me | OCONMe₂ | H | H | OCONMe₂ | — |
| 22 | S | S | t-Bu | OCON(n-C₆H₁₃)₂ | H | H | OCON(n-C₆H₁₃)₂ | — |
| 23 | S | S | t-Bu | Set | H | H | SEt | — |
| 24 | S | S | t-Bu | F | F | F | F | — |
| 25 | S | S | Me | NEt₂ | H | H | NEt₂ | — |
| 26 | S | S | t-Bu | OH | H | H | OH | — |
| 27 | S | S | t-Bu | OH | H | H | OCOCH(n-Bu)Et | — |
| 28 | S | S | t-Bu | OH | COCH₃ | H | OMe | — |
| 29 | S | S | Ph | OH | H | H | OCH₂CH(n-Bu)Et | — |
| 30 | S | S | t-Bu | OMe | CN | H | OMe | — |

TABLE 2

| Compound | | | | | | | | |
|---|---|---|---|---|---|---|---|---|
| No. | X¹ | X² | R¹¹ | R¹² | R¹³ | R¹⁴ | R¹⁵ | R¹⁶ |
| 31 | S | S | t-Bu | OTs | H | H | OTs | — |
| 32 | S | S | t-Bu | OMs | H | H | OMs | — |
| 33 | S | S | t-Bu | CN | H | H | CN | — |
| 34 | S | S | t-Bu | H | H | H | H | — |
| 35 | S | S | Me | H | H | H | H | — |
| 36 | S | S | i-Pr | H | H | H | H | — |
| 37 | S | S | Ph | H | H | H | H | — |
| 38 | S | S | t-Bu | H | Cl | H | H | — |
| 39 | S | S | t-Bu | H | t-Bu | H | H | — |
| 40 | S | S | Me | H | t-Bu | H | H | — |
| 41 | NR¹⁶ | S | t-Bu | H | H | H | H | Me |
| 42 | NR¹⁶ | S | t-Bu | H | H | H | H | Et |
| 43 | NR¹⁶ | S | t-Bu | H | H | H | H | n-Hex |
| 44 | NR¹⁶ | S | Me | H | H | H | H | n-Hex |
| 45 | NR¹⁶ | S | Ph | H | H | H | H | n-Hex |
| 46 | NR¹⁶ | S | t-Bu | H | H | OMe | H | n-Hex |
| 47 | NR¹⁶ | S | t-Bu | H | OMe | H | H | Me |
| 48 | NR¹⁶ | S | t-Bu | H | Me | Me | H | n-Hex |
| 49 | NR¹⁶ | S | t-Bu | H | H | t-Bu | H | Me |
| 50 | NR¹⁶ | S | Ph | H | H | Me | H | n-Hex |
| 51 | NR¹⁶ | S | t-Bu | H | H | CN | H | n-Hex |
| 52 | NR¹⁶ | S | t-Bu | H | H | Cl | H | n-Hex |
| 53 | NR¹⁶ | S | t-Bu | H | H | H | H | CH₂Ph |
| 54 | NR¹⁶ | O | t-Bu | H | H | H | H | n-Hex |
| 55 | NR¹⁶ | O | t-Bu | H | H | H | H | Me |
| 56 | NR¹⁶ | O | Me | H | H | H | H | n-Hex |
| 57 | NR¹⁶ | O | Me | H | H | Me | H | n-Hex |
| 58 | NR¹⁶ | O | Ph | H | H | H | H | n-Hex |
| 59 | NR¹⁶ | NR¹⁶ | t-Bu | H | H | H | H | n-Hex |
| 60 | NR¹⁶ | NR¹⁶ | t-Bu | H | H | H | H | Me |
| 61 | NR¹⁶ | NR¹⁶ | t-Bu | H | H | H | H | Et |

The exemplified compounds (1) to (5), (7) to (10), (12) to (19), (21) to (28), (30) to (36), and (38) to (40) are included in not only the compound represented by formula (1) but also the compound represented by formula (2).

These compounds may be synthesized in accordance with known synthetic methods of similar compounds. These compounds can be synthesized according to any one of the methods described or cited, for example in Journal of Chemical Crystallography, 27, 1997, p. 516, right column, line 3 to p. 520, right column, line 15; Liebigs Annalen der Chemie, 726, p. 106, line 15 to p. 109, line 37; JP-A-49-1115, p. 3, left column, line 7 to p. 5, left column, line 8; Bioorganic & Medicinal Chemistry Letters, 7, 1997, p. 652, lines 9 to 19; Journal of Organic Chemistry, 43, 1978, p. 2153, left column, lines 2 to 12; JP-A-4-338759, p. 4, left column, line 2 to p. 5, left column, line 2; JP-A-3-54566, p. 7, left column, line 6 to p. 8, left column, line 10; Synthesis, 1986, p. 968, left column, lines 1 to 22, or a method similar to that.

Specifically, the compound represented by formula (1) can be obtained by reacting, for example, a benzodithiol or benzothiazole compound having a nitrogen-elimination group, a sulfur-elimination group, or an oxygen-elimination group, that is represented by the following formula (3) or (4) and an isooxazolone compound as represented by the following formula (5). More specifically, details will be described in Examples.

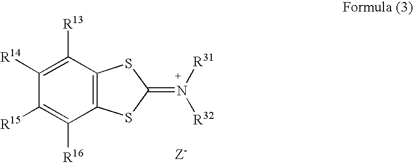

In formula (3), R¹³, R¹⁴, R¹⁵ and R¹⁶ each have the same meanings as those of R¹³, R¹⁴, R¹⁵, and R¹⁶ in formula (1), and a preferable range of each of R¹³, R¹⁴, R¹⁵, and R¹⁶ is the same as those of R¹³, R¹⁴, R¹⁵, and R¹⁶ in formula (1). Z represents a counter anion. R³¹ and R³² each independently represent a substituted or unsubstituted alkyl group. R³¹ and R³² may combine with each other to form a ring.

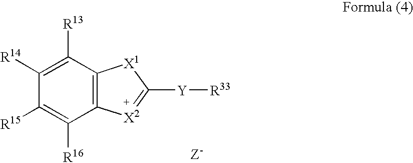

In formula (4), X¹, X², R¹³, R¹⁴, R¹⁵, and R¹⁶ each have the same meanings as) those of X¹, X², R¹³, R¹⁴, R¹⁵, R¹⁶ in formula (1), and a preferable range of each of X¹, X², R¹³, R¹⁴, R¹⁵ and R¹⁶ is the same as those of X¹, X², R¹³, R¹⁴, R¹⁵, and R¹⁶ in formula (1). Z represents a counter anion. Y represents a sulfur ion or an oxygen ion. R³³ represents a substituted or unsubstituted alkyl group, or a substituted or unsubstituted aryl group.

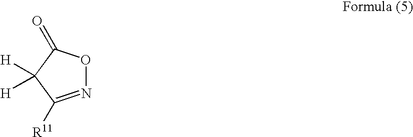

In formula (5), R¹¹ has the same meaning as that of R¹¹ in formula (1), and a preferable range of R¹¹ is the same as that of R¹¹ in formula (1).

The compound represented by the formula (3) can be synthesized in accordance with a method described in, for example, Angew. Chem. Int. Ed., 42, 24 (2003), p. 2765.

The compound represented by the formula (4) can be synthesized in accordance with a method described in, for example, J. Prakt. Chem., 325, 5 (1983), p. 811 or J. Chem. Soc., (1958) p. 854.

Among the compounds represented by the formula (5), for example, a synthesis of the compound wherein R¹¹ is a t-butyl group is described in J. Mater. Chem., 11 (2001), page 2277, line 19, et seq. The compound wherein R¹¹ is a group other than the t-butyl group can be also synthesized in accordance with a method similar to the above-described synthetic method.

The compound represented by formula (1) or (2) is preferably used as a long-wavelength ultraviolet absorbent. The maximum absorption wavelength of the compound is preferably in the range of 300 nm to 400 nm, more preferably from 340 nm to 390 nm, and furthermore preferably from 360 nm to 390 nm.

The molar extinction coefficient of the compound represented by the formula (1) or (2) in the maximum absorption wavelength is preferably 20,000 or more, and especially preferably 23,000 or more. If the molar extinction coefficient is less than 20,000, absorption efficiency of the ultraviolet absorbent per mass gets worse. As a result, it is necessary to use large amounts of the ultraviolet absorbent in order to absorb ultraviolet rays in its entire range. This is not preferable because work efficiency gets worse and also possibility of bleed-out increases, or for other reasons.

The absorption maximum wavelength of the ultraviolet absorbent compounds in the present invention are determined by preparing a solution in ethyl acetate as the solvent at a concentration of approximately 10×10⁻⁶ mol·dm⁻³ and by measurement while using a quartz cell having an optical path length of 1 cm. The molar extinction coefficient is defined, for example, in Chemical Society of Japan Ed., “New Experimental Chemistry Lecture, Chapter 9 Analytical Chemistry [II]”, (Maruzen, 1977), p. 244, and can be determined, together with the absorption maximum wavelength.

The polymer composition is used in preparation of the polymer material according to the present invention. The polymer composition for use in the present invention contains a polymer substance described below, and the ultraviolet absorbent comprising the compound represented by formula (1) or (2).

The ultraviolet absorbent comprising the compound represented by formula (1) or (2) may be contained in the polymer substance in various methods. When the ultraviolet absorbent comprising the compound represented by formula (1) or (2) is compatible with the polymer substance, the ultraviolet absorbent comprising the compound represented by formula (1) or (2) may be added to the polymer substance directly. The ultraviolet absorbent comprising the compound represented by formula (1) or (2) may be dissolved in a cosolvent compatible with the polymer substance, and then the obtained solution be added to the polymer substance. The ultraviolet absorbent comprising the compound represented by formula (1) or (2) may be dispersed in a polymer, and the obtained dispersion be added to the polymer substance.

Regarding the method of adding the ultraviolet absorbent comprising the compound represented by formula (1) or (2) to the polymer substance, reference can be made to the description in JP-A-58-209735, JP-A-63-264748, JP-A-4-191851, JP-A-8-272058, and British Patent No. 2016017A.

In the present invention, two or more kinds of the ultraviolet absorbents comprising the compounds represented by formula (1) or (2) different in structure each other may be used in combination. Alternatively, the ultraviolet absorbent of the present invention and one or more kinds of ultraviolet absorbents different in structure may be used in combination. Two kinds (preferably three kinds) of ultraviolet absorbents when used in combination absorb ultraviolet ray in a wider wavelength range. In addition, the use of two or more kinds of ultraviolet absorbents in combination has a function to stabilize the dispersion state.

Any ultraviolet absorbent having a chemical structure other than that of ultraviolet absorbent in the present invention may be used. Examples thereof include those described, for example, in Yasuichi Okatsu Ed., “Development of Polymer Additives and Environmental Measures” (CMC Publishing, 2003), Chapter 2; and Toray Research Center Inc., Technical Survey Dept., Ed., “New Trend of Functional Polymer Additives” (Toray Research Center Inc., 1999), Chapter 2.3.1. Examples thereof include ultraviolet absorbing structures such as triazine-based, benzotriazole-based, benzophenone-based, merocyanine-based, cyanine-based, dibenzoylmethane-based, cinnamic acid-based, acrylate-based, benzoic ester-based, and oxalic diamide-based compounds. Specific examples thereof are described, for example, in Fine Chemicals, 2004, May, p. 28 to 38; Toray Research Center Inc., Technical Survey Dept., Ed., “New Trend of Functional Polymer Additives” (Toray Research Center Inc., 1999), p. 96 to 140; and Yasuichi Okatsu Ed., “Development of Polymer Additives and Environmental Measures” (CMC Publishing, 2003), p. 54 to 64.

Among these, preferable are benzotriazole-based, benzophenone-based, salicylic acid-based, acrylate-based, and triazine-based compounds. More preferable are benzotriazole-based, benzophenone-based, and triazine-based compounds. Particularly preferable are benzotriazole-based and triazine-based compounds.

The benzotriazole-based compounds preferably has the effective absorption wavelength of approximately 270 to 380 nm, and specific examples thereof include 2-(2′-hydroxy-5′-methylphenyl)benzotriazole, 2-(2′-hydroxy-5′-t-butylphenyl)benzotriazole, 2-(2′-hydroxy-3′-t-butyl-5′-methylphenyl)-5-chlorobenzotriazole, 2-(2′-hydroxy-3′,5′-di-t-butylphenyl)-5-chlorobenzotriazole, 2-(2′-hydroxy-3′-dodecyl-5′-methylphenyl)-5-chlorobenzotriazole, 2-(2′-hydroxy-3′,5′-di-t-amylphenyl)benzotriazole, 2-(2′-hydroxy-5′-(1,1,3,3-tetramethylbutyl)phenyl)benzotriazole, 2-(2′-hydroxy-4′-octyloxyphenyl)benzotriazole, 2-(2′-hydroxy-3′-(3,4,5,6-tetrahydrophthalimidylmethyl)-5′-methylbenzyl)phenyl)benzotriazole, 2-(3′-sec-butyl-5′-t-butyl-2′-hydroxyphenyl)benzotriazole, 2-(3′,5′-bis-(α,α-dimethylbenzyl)-2′-hydroxyphenyl)benzotriazole, 2-(3′-t-butyl-2′-hydroxy-5′-(2-octyloxycarbonylethyl)phenyl)-5-chloro-benzotriazole, 2-(3′-t-butyl-5′-[2-(2-ethylhexyloxy)-carbonylethyl]-2′-hydroxyphenyl)-5-chloro-benzotriazole, 2-(3′-t-butyl-2′-hydroxy-5′-(2-methoxycarbonylethyl)phenyl)-5-chloro-benzotriazole, 2-(3′-t-butyl-2′-hydroxy-5′-(2-methoxycarbonylethyl)phenyl)benzotriazole, 2-(3′-t-butyl-2′-hydroxy-5′-(2-octyloxycarbonylethyl)phenyl)benzotriazole, 2-(3′-t-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-t-butyl-2′-hydroxy-5′-(2-isooctyloxycarbonylethyl)phenylbenzotriazole, 2,2′-methylene-bis[4-(1,1,3,3-tetramethylbutyl)-6-benzotriazole-2-ylphenol], and the like.

The triazine-based compounds preferably has the effective absorption wavelength of approximately 270 to 380 nm, and specific examples thereof include 2-(4-butoxy-2-hydroxyphenyl)-4,6-di(4-butoxyphenyl)-1,3,5-triazine, 2-(4-butoxy-2-hydroxyphenyl)-4,6-di(2,4-dibutoxyphenyl)-1,3,5-triazine, 2,4-di(4-butoxy-2-hydroxyphenyl)-6-(4-butoxyphenyl)-1,3,5-triazine, 2,4-di(4-butoxy-2-hydroxyphenyl)-6-(2,4-dibutoxyphenyl)-1,3,5-triazine, 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris(2-hydroxy-4-(3-butoxy-2-hydroxypropoxy)phenyl)-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxy-propyloxy]phenyl}-4,6-bis(2,4-dimethylphenyl)-1,3,5-triazine and 2-(2-hydroxy-4-(2-ethylhexyl)oxy)phenyl-4,6-di(4-phenyl)phenyl-1,3,5-triazine.

The benzophenone-based compounds preferably has the effective absorption wavelength of approximately 270 to 380 mm, and specific examples thereof include 2,4-dihydroxybenzophenone, 2-hydroxy-4-methoxybenzophenone, 2-hydroxy-4-octyloxybenzophenone, 2-hydroxy-4-decyloxybenzophenone, 2-hydroxy-4-dodecyloxybenzophenone, 2-hydroxy-4-benzyloxybenzophenone, 2-hydroxy-4-(2-hydroxy-3-methacryloxypropoxy)benzophenone, 2-hydroxy-4-methoxy-5-sulfobenzophenone, 2-hydroxy-4-methoxy-5-sulfobenzophenone trihydrate, 2-hydroxy-4-methoxy-2′-carboxybenzophenone, 2-hydroxy-4-octadecyloxybenzophenone, 2-hydroxy-4-diethylamino-2′-hexyloxycarbonylbenzophenone, 2,2′-dihydroxy-4-methoxybenzophenone, 2,2′,4,4′-tetrahydroxybenzophenone, 2,2′-dihydroxy-4,4′-dimethoxybenzophenone, and 1,4-bis(4-benzyloxy-3-hydroxyphenoxy)butane.

The salicylic acid-based compound above is preferably a compound having an effective absorption wavelength of approximately 290 to 330 nm, and typical examples thereof include phenyl salicylate, 4-t-butylphenyl salicylate, 4-octylphenyl salicylate, dibenzoylresorcinol, bis(4-t-butylbenzoyl)resorcinol, benzoylresorcinol, 2,4-di-t-butylphenyl 3,5-di-t-butyl-4-hydroxysalicylate, and hexadecyl 3,5-di-t-butyl-4-hydroxysalicylate.

The acrylate-based compound above is preferably a compound having an effective absorption wavelength of approximately 270 to 350 nm, and typical examples thereof include 2-ethylhexyl 2-cyano-3,3-diphenylacrylate, ethyl 2-cyano-3,3-diphenylacrylate, isooctyl 2-cyano-3,3-diphenylacrylate, hexadecyl 2-cyano-3-(4-methylphenyl)acrylate, methyl 2-cyano-3-methyl-3-(4-methoxyphenyl)cinnamate, butyl 2-cyano-3-methyl-3-(4-methoxyphenyl)cinnamate, methyl 2-carbomethoxy-3-(4-methoxyphenyl)cinnamate 2-cyano-3-(4-methylphenyl)acrylate salt, 1,3-bis(2′-cyano-3,3′-diphenylacryloyl)oxy)-2,2-bis(((2′-cyano-3,3′-diphenylacryloyl)oxy)methyl)propane, and N-(2-carbomethoxy-2-cyanovinyl)-2-methylindoline.

The oxalic diamide-based compound above is preferably a compound having an effective absorption wavelength of approximately 250 to 350 nm, and typical examples thereof include 4,4′-dioctyloxyoxanilide, 2,2′-dioctyloxy-5,5′-di-t-butyloxanilide, 2,2′-didodecyloxy-5,5′-di-t-butyloxanilide, 2-ethoxy-2′-ethyloxanilide, N,N′-bis(3-dimethylaminopropyl)oxamide, 2-ethoxy-5-t-butyl-2′-ethyloxanilide, and 2-ethoxy-2′-ethyl-5,4′-di-t-butyloxanilide.

The polymer material (ultraviolet absorbent composition) of the present invention may further contain a light stabilizer, or an antioxidant.

Preferable examples of the light stabilizer and the antioxidant include compounds described in JP-A-2004-117997. Specifically, compounds described on page 29, middle paragraph Nos. [0071] to [0111] of JP-A-2004-117997 are preferable; and especially, compounds represented by formula (TS-I), (TS-II), (TS-IV), or (TS-V) described on the paragraph No. [0072] are preferable.

The content of the ultraviolet absorbent comprising the compound represented by formula (1) or (2), in the polymer material of the present invention, may vary according to the application and the usage of the polymer material and thus cannot be defined specifically, but can be determined easily by the person skilled in the art after some tests. It is preferably 0.001 to 10 mass %, more preferably 0.01 to 5 mass %, with respect to the total amount of the polymer material. The content of the ultraviolet absorbent other than ultraviolet absorbent comprising the compound represented by formula (1) or (2) above can be determined properly according to the application of the present invention.

Although practically sufficient ultraviolet-shielding effect is obtained only with the ultraviolet absorbent in the present invention, a white pigment which has higher hiding power such as titanium oxide may be used for assurance. In addition, a trace (e.g. 0.05 mass % or less) amount of colorant may be used additionally, if the appearance or the color tone is of a problem or as needed. Alternatively, a fluorescent brightener may be used additionally for applications demanding transparency or whiteness. Examples of the fluorescent brighteners include commercialized products, the compounds described in JP-A-2002-53824, and the like.

Hereinafter, the polymer substance that can be used in the polymer material of the present invention will be described. An acrylic acid-based polymer, a polyester, a polycarbonate, or the blend thereof is preferably used as the polymer substance. Hereinafter, each of the polymers will be described in detail.

### [Acrylic Acid-Based Polymer]

The acrylic acid-based polymer, as used herein, is preferably a homopolymer or a copolymer obtained by polymerization of a compound represented by formula (A1) as the monomer component.

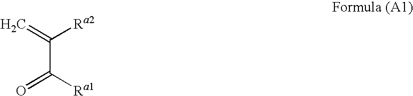

(In formula (A1), R^{a1} represents a hydroxyl group, a substituted or unsubstituted alkoxy group, a substituted or unsubstituted amino group, a substituted or unsubstituted alkyl group, a substituted or unsubstituted aryl group, or a substituted or unsubstituted heterocyclic group; R^{a2} represents a hydrogen atom, a methyl group, or an alkyl group having 2 or more carbon atoms.)

The formula (A1) will be described in detail.

In formula (A1), R^{a1} represents a hydroxyl group, a substituted or unsubstituted alkoxy group, a substituted or unsubstituted aryloxy group, a substituted or unsubstituted amino group, a substituted or unsubstituted alkyl group, a substituted or unsubstituted aryl group, or a substituted or unsubstituted heterocyclic group. Among these, is preferably a substituted or unsubstituted alkoxy group, or a substituted or unsubstituted aryloxy group; and particularly preferably a substituted or unsubstituted alkoxy group having 1 to 18 carbon atoms, or a substituted or unsubstituted aryloxy group having 6 to 24 carbon atoms.

R^{a2} represents a hydrogen atom, a methyl group, or an alkyl group having 2 or more carbon atoms. Among these, R^{a2} is preferably a hydrogen atom or a methyl group.

Thus, in preferable combination of the substituents of formula (A1), Rat represents a substituted or unsubstituted alkoxy group having 1 to 18 carbon atoms or a substituted or unsubstituted aryloxy group having 6 to 24 carbon atoms, and R^{a2} represents a hydrogen atom or a methyl group.

Typical examples of the compound represented by formula (A1) include the followings:

acrylate derivatives such as methyl acrylate, ethyl acrylate, (n- or i-)propyl acrylate, (n-, i-, sec- or t-)butyl acrylate, amyl acrylate, 2-ethylhexyl acrylate, dodecyl acrylate, chloroethyl acrylate, 2-hydroxyethyl acrylate, 2-hydroxypropyl acrylate, 2-hydroxypentyl acrylate, cyclohexyl acrylate, allyl acrylate, trimethylolpropane monoacrylate, pentaerythritol monoacrylate, benzyl acrylate, methoxybenzyl acrylate, chlorobenzyl acrylate, hydroxybenzyl acrylate, hydroxyphenethyl acrylate, dihydroxyphenethyl acrylate, furfuryl acrylate, tetrahydrofurfuryl acrylate, phenyl acrylate, hydroxyphenyl acrylate, chlorophenyl acrylate, sulfamoylphenyl acrylate, and 2-(hydroxyphenylcarbonyloxy)ethyl acrylate;
methacrylate derivatives such as methyl methacrylate, ethyl methacrylate, (n- or i-) propyl methacrylate, (n-, i-, sec- or t-)butyl methacrylate, amyl methacrylate, 2-ethylhexyl methacrylate, dodecyl methacrylate, chloroethyl methacrylate, 2-hydroxyethyl methacrylate, 2-hydroxypropyl methacrylate, 2-hydroxypentyl methacrylate, cyclohexyl methacrylate, allyl methacrylate, trimethylolpropane monomethacrylate, pentaerythritol monomethacrylate, benzyl methacrylate, methoxybenzyl methacrylate, chlorobenzyl methacrylate, hydroxybenzyl methacrylate, hydroxyphenethyl methacrylate, dihydroxyphenethyl methacrylate, furfuryl methacrylate, tetrahydrofurfuryl methacrylate, phenyl methacrylate, hydroxyphenyl methacrylate, chlorophenyl methacrylate, sulfamoylphenyl methacrylate, and 2-(hydroxyphenylcarbonyloxy)ethyl methacrylate; acrylamide derivatives such as acrylamide, N-methylacrylamide, N-ethylacrylamide, N-propylacrylamide, N-butylacrylamide, N-benzylacrylamide, N-hydroxyethylacrylamide, N-phenylacrylamide, N-tolylacrylamide, N-(hydroxyphenyl)acrylamide, N-(sulfamoylphenyl)acrylamide, N-(phenylsulfonyl)acrylamide, N-(tolylsulfonyl)acrylamide, N,N-dimethylacrylamide, N-methyl-N-phenylacrylamide, and N-hydroxyethyl-N-methylacrylamide; and methacrylamide derivatives such as methacrylamide, N-methylmethacrylamide, N-ethylmethacrylamide, N-propylmethacrylamide, N-butylmethacrylamide, N-benzylmethacrylamide, N-hydroxyethylmethacrylamide, N-phenylmethacrylamide, N-tolylmethacrylamide, N-(hydroxyphenyl)methacrylamide, N-(sulfamoylphenyl)methacrylamide, N-(phenylsulfonyl)methacrylamide, N-(tolylsulfonyl)methacrylamide, N,N-dimethylmethacrylamide, N-methyl-N-phenylmethacrylamide, and N-hydroxyethyl-N-methylmethacrylamide.

The acrylic acid-based polymer is preferably a single-component homopolymer obtained by polymerization of the monomer represented by formula (A1) above or a two-, three- or four-component, more preferably two- or three-component, copolymer prepared by polymerization using the monomer represented by formula (A1) above at a molar ratio of 10% to 90%, preferably 20% to 80% and also other monomer components or the other monomer components represented by formula (A1) above. Examples of the aforementioned other monomer components include a substituted or unsubstituted styrene derivative, and acrylonitrile.

The acrylic acid-based polymer is preferably a homopolymer containing an acrylate or a methacrylate having 4 to 24 carbon atoms as the repeating unit or a two- or three-component copolymer containing an acrylate or a methacrylate as the repeating unit at a molar ratio of 10% to 90%.

### [Polyester]

Hereinafter, the polyester will be described. The polyester that can be used in the present invention contains the following dicarboxylic acid, the acid halide thereof or the following polyvalent carboxylic acid; and a diol as monomer components.

Examples of the dicarboxylic acid or the acid halides thereof include aliphatic or, alicyclic dicarboxylic acids such as adipic acid, superic acid, azelaic acid, sebacic acid, dodecanedioic acid, oxalic acid, malonic acid, succinic acid, glutaric acid, ethylsuccinic acid, pimelic acid, maleic acid, fumaric acid, itaconic acid, citraconic acid, mesaconic acid, 2-methylsuccinic acid, 2-methyladipic acid, 3-methyladipic acid, 3-methylpentanedioic acid, 2-methyloctanedioic acid, 3,8-dimethyldecanedioic acid, 3,7-dimethyldecanedioic acid, dimer acid, hydrogenated dimer acids, 1,2- or 1,3-cyclopentanedicarboxylic acids, and 1,2-, 1,3- or 1,4-cyclohexanedicarboxylic acids; and

aromatic dicarboxylic acids such as phthalic acid, isophthalic acid, terephthalic acid, 1,4-naphthalenedicarboxylic acid, 2,5-naphthalenedicarboxylic acid, 2,6-naphthalenedicarboxylic acid, naphthalic acid, biphenyldicarboxylic acid, 2-methylisophthalic acid, 3-methylphthalic acid, 2-methylterephthalic acid, 2,4,5,6-tetramethylisophthalic acid, 3,4,5,6-tetramethylphthalic acid, 2-chloroterephthalic acid, 2-methylterephthalic acid, 5-methylisophthalic acid, 5-sodium sulfoisophthalic acid, 2,6-naphthalenedicarboxylic acid, hexahydroterephthalic acid, hexahydroisophthalic acid, 3-chloroisophthalic acid, 3-methoxyisophthalic acid, 2-fluoroisophthalic acid, 3-fluorophthalic acid, 2-fluoroterephthalic acid, 2,4,5,6-tetrafluoroisophthalic acid, 3,4,5,6-tetrafluorophthalic acid, 4,4′-oxybisbenzoic acid, 3,3′-oxybisbenzoic acid, 3,4′-oxybisbenzoic acid, 2,4′-oxybisbenzoic acid, 3,4′-oxybisbenzoic acid, 2,3′-oxybisbenzoic acid, 4,4′-oxybisoctafluorobenzoic acid, 3,3′-oxybisoctafluorobenzoic acid, 1,4-naphthalenedicarboxylic acid, 2,6-naphthalenedicarboxylic acid, 4,4′-biphenyldicarboxylic acid, 4,4′-diphenylethercarboxylic acid; and the like.

Examples of the polyvalent carboxylic acids other than the dicarboxylic acids include ethanetricarboxylic acid, propanetricarboxylic acid, butanetetracarboxylic acid, pyromellitic acid, trimellitic acid, trimesic acid, and 3,4,3′,4′-biphenyltetracarboxylic acid.

With respect to the polyester that can be used in the present invention, among these dicarboxylic acids and polyvalent carboxylic acid components, use of adipic acid, malonic acid, succinic acid, terephthalic acid, isophthalic acid, phthalic acid, 1,4-naphthalenedicarboxylic acid, 2,5-naphthalenedicarboxylic acid, 2,6-naphthalenedicarboxylic acid or trimellitic acid is preferable; and use of terephthalic acid, isophthalic acid, 1,4-naphthalenedicarboxylic acid, 2,5-naphthalenedicarboxylic acid or 2,6-naphthalenedicarboxylic acid is particularly preferable.

Examples of the diols include aliphatic glycols such as ethylene glycol, 1,2-propylene glycol, 1,3-propylene glycol, diethylene glycol, triethylene glycol, 1,2-butylene glycol, 1,3-butylene glycol, 2,3-butylene glycol, 1,4-butylene glycol, 1,5-pentanediol, neopentylglycol, 1,6-hexanediol, 1,2-cyclohexanediol, 1,3-cyclohexanediol, 1,4-cyclohexanediol, 1,2-cyclohexanedimethanol, 1,3-cyclohexanedimethanol, 1,4-cyclohexanedimethanol, 1,4-cyclohexanediethanol, 1,10-decamethylene glycol, 1,12-dodecanediol, polyethylene glycol, polytrimethylene glycol, and polytetramethylene glycol; aromatic glycols such as hydroquinone, 4,4′-dihydroxybisphenol, 1,4-bis(β-hydroxyethoxy)benzene, 1,4-bis(β-hydroxyethoxyphenyl)sulfone, bis(p-hydroxyphenyl)ether, bis(p-hydroxyphenyl)sulfone, bis(p-hydroxyphenyl)methane, 1,2-bis(p-hydroxyphenyl)ethane, bisphenol A, bisphenol C, 2,5-naphthalenediol, and ethyleneoxide adducts of these glycols; and the like.

With respect to the polyester that can be used in the present invention, among these diol components, use of ethylene glycol, 1,3-propylene glycol, diethylene glycol, neopentylglycol, hydroquinone, 4,4′-dihydroxybisphenol or bisphenol A is preferable; and use of ethylene glycol or 4,4′-dihydroxybisphenol is particularly preferable.

Specifically, preferable combinations of monomers and preferable polymers in the polyester that can be used in the present invention include polyethylene terephthalate prepared by using terephthalic acid as the dicarboxylic acid component and ethylene glycol as the diol component, polybutylene terephthalate prepared by using terephthalic acid as the dicarboxylic acid component and 1,4-butylene glycol as the diol component, and polyethylene naphthalate prepared by using 2,6-naphthalenedicarboxylic acid as the dicarboxylic acid component and ethylene glycol as the diol component.

### [Polycarbonate]

The polycarbonate that can be used in the present invention is prepared from the following polyvalent phenols and the following carbonates such as bisalkyl carbonate, bisaryl carbonate or phosgene.

Examples of the polyvalent phenols include hydroquinone, resorcin, 4,4′-dihydroxydiphenyl, bis(4-hydroxyphenyl)methane, 1,1-bis(4-hydroxyphenyl)ethane, 1,1-bis(4-hydroxyphenyl)-1-phenylethane, bisphenol A, bisphenol C, bisphenol E, bisphenol F, bisphenol M, bisphenol P, bisphenol S, bisphenol Z, 2,2-bis(3-methyl-4-hydroxyphenyl)propane, 1,1-bis(4-hydroxyphenyl)cyclohexane, 2,2-bis(3-phenyl-4-hydroxyphenyl)propane, 2,2-bis(3-isopropyl-4-hydroxyphenyl)propane, 2,2-bis(4-hydroxyphenyl)butane, 2,2-bis(3,5-dimethyl-4-hydroxyphenyl)propane, 2,2-bis(3,5-dibromo-4-hydroxyphenyl)propane, 4,4′-dihydroxydiphenylsulfone, 4,4′-dihydroxydiphenylsulfoxide, 4,4′-dihydroxydiphenylsulfide, 3,3′-dimethyl-4,4′-dihydroxydiphenylsulfide, and 4,4′-dihydroxydiphenyloxide.

With respect to the polycarbonate that can be used in the present invention, among these polyvalent phenol components, use of hydroquinone, resorcin, 4,4′-dihydroxydiphenyl or bisphenol A is preferable.

Examples of the carbonates include phosgene, diphenyl carbonate, bis(chlorophenyl)carbonate, dinaphthyl carbonate, bis(diphenyl)carbonate, dimethyl carbonate, diethyl carbonate, and dibutyl carbonate.

With respect to the polycarbonate that can be used in the present invention, among these carbonate components, use of phosgene, bis(diphenyl)carbonate, dimethyl carbonate, or diethyl carbonate is preferable.

Specifically, a preferable combination of monomers, i.e., a preferable polymer in the polycarbonate that can be used in the present invention is bisphenol A carbonate, which is prepared by using bisphenol A as the polyvalent phenol component and phosgene as the carbonate component.

Among the polymers above, polymethyl acrylate, polymethyl methacrylate, polyethylene terephthalate, polyethylene naphthalate, polybutylene terephthalate and polycarbonate are particularly preferable. Unexpectedly to the person skilled in the art, use of one of the preferable polymer substances resulted in drastic improvement in light fastness of the ultraviolet absorbent, compared to the ultraviolet absorbent prepared with a polymer substance other than those above.

The polymer substance for use in the present invention is preferably a thermoplastic resin.

The polymer substance for use in the present invention preferably has a transmittance of 80% or more. The transmittance in the present invention is the total light transmittance as determined according to the method described in the Chemical Society of Japan Ed., “Experimental Chemistry Lecture 29—Polymer materials,” 4th Ed., (Maruzen, 1992) p. 225 to 232.

The glass transition point (Tg) of the polymer substance for use in the present invention is preferably −80° C. or higher and 200° C. or lower, still more preferably −30° C. or higher and 180° C. or lower. In particular, a polyacrylate, a polycarbonate and a polyethylene terephthalate are preferable.

The polymer material prepared by using a polymer substance having a Tg in the range above gives a polymer material favorably in flexibility and hardness. When a polyacrylate, polycarbonate or polyethylene terephthalate is used, it leads to improvement in operational efficiency; and when the ultraviolet absorbent comprising the compound represented by formula (1) or (2) is used, it leads to improvement in the light fastness of the ultraviolet absorbent itself.

The polymer material according to the present invention may contain any additives such as antioxidant, photostabilizer, processing stabilizer, antidegradant, and compatibilizer, as needed in addition to the polymer substance above and the ultraviolet absorbent.

The polymer material according to the present invention contains the polymer substance above. The polymer material according to the present invention may be made only of the above-described polymer substance, or may be formed by using the polymer substance dissolved in a solvent.

When the polyethylene terephthalate is used as the polymer substance, the polymer material according to the present invention is preferably produced by melt-kneading of the polyethylene terephthalate and the ultraviolet absorbent at a temperature of 200° C. or higher. Polymer materials prepared by the melt-kneading of polyethylene terephthalate at the temperature or less possibly may give polymer materials containing the ultraviolet absorbent unevenly dispersed in the spot-like pattern.

The content of the ultraviolet absorbent in the polymer material according to the present invention is preferably 0.1 mass % to 50 mass %, more preferably 0.1 mass % to 25 mass %, and particularly preferably 0.4 mass % to 10 mass %, with respect to 100 mass % of the polyethylene terephthalate. A content of not more than 0.1 mass % may result in production of a polymer material that does not absorb the light in the ultraviolet region completely, because of insufficiency of the ultraviolet absorbent added.

The compound represented by formula (1) or (2) for use in the present invention, which is superior in solubility, gives a polymer material easily, as it is dissolved in a various solvent with a polymer and the solution coated. In preparation of the polymer material, a plasticizer may not be added. In addition, a polymer material prepared by solvent coating or polymer kneading has an advantage that it is superior in light fastness, compared to the polymer material prepared by using a plasticizer.

The compound represented by formula (1) or (2) mostly have a molecular weight of 1000 or less, and thus, the idea of using such a compound as it is melted under an environment at high temperature for prolonged period, for example during PET kneading, which may lead to volatilization and decomposition, was not easily conceived by the person skilled in the art.

When the acrylate or the polycarbonate is used as the polymer substance, the polymer material according to the present invention is preferably prepared by dissolving the acrylate or the polycarbonate and the ultraviolet absorbent in a solvent having a boiling point of 200° C. or lower and applying the resulting solution on a base substrate. If a solvent having a boiling point higher than 200° C. is used in applying the ultraviolet absorbent, it is needed to volatilize the solvent at a higher temperature, which may make the processing step more complicated.

The content of the ultraviolet absorbent in the polymer material according to the present invention, is preferably 0.1 mass % to 50 mass %, more preferably 0.1 mass % to 25 mass %, and particularly more preferably 0.4 mass % to 10 mass %, with respect to 100 mass % of the acrylate or the polycarbonate. When the added amount is 0.1 mass % or less, polymer materials absorbing the light in the entire ultraviolet region may not be produced, because of insufficiency of the ultraviolet absorbent added.

The solvent having a boiling point of 200° C. or less that can be used in the present invention is not particularly limited, as long as the solvent is able to dissolve or disperse the ultraviolet absorbent of the present invention. The boiling point of the solvent is preferably in the range of 0° C. to 200° C., more preferably from 20° C. to 150° C., and furthermore preferably from 30° C. to 120° C., from viewpoints of the coated surface state and drying of the solvent after coating. Examples of the solvent include alcoholic solvents (e.g., methanol, ethanol, isopropanol, and tetrafluoropropanol), halogen-series solvents (e.g., methylene chloride, chloroform, chlorobenzene, and dichlorobenzene), ketone-series solvents (e.g., acetone, ethylmethylketone, and cyclohexanone), hydrocarbon-series solvents (e.g., benzene, toluene, and cyclohexane), ester-series solvents (e.g., ethyl acetate, and butyl acetate), and ether-series solvents (e.g., dioxane, and tetrahydrofuran). If necessary, these solvents may be used in combination of two or more kinds.

Examples of the substrate that can be used in the present invention include inorganic substrates such as a glass substrate, an iron substrate, an aluminum substrate, a silicon substrate, and a ceramic substrate; and polymer material substrates such as a polyethylene terephthalate (PET) film substrate, a triacetyl cellulose (TAC) film substrate, or a polycarbonate film substrate. The form of these substrates may be various forms such as a plate-like, sheet-like, or disc-like shape. That is, any shape of substrate may be used, as long as the shape does not prevent a polymer material from being coated.

The polymer material according to the present invention is applicable to any application where synthetic resin is used, and particularly favorably to applications where there is possibility of exposure to light such as sunlight or ultraviolet light. Specific examples thereof include glass alternatives and their surface-coating material; coating agents for the window glass, lighting glass and light source-protecting glass such as of house, facility, and vehicle; interior and exterior materials such as of house, facility and vehicle, paints for the interior and exterior materials; materials for ultraviolet-emission sources such as fluorescent lamp and mercury lamp; materials for precision machines and electric and electronic devices; materials for shielding electromagnetic and other waves emitted from various displays; containers and packaging materials such as of food, chemicals, and medicine; discoloration inhibitors for agricultural and industrial sheet or film, print, colored products, dyes and pigments; cosmetics such as anti-sunburn cream, shampoo, rinse, and hair dressing; apparel fiber products such as sport wear, stockings and cap and the fibers; home interior products such as curtain, carpet and wall paper; medical devices such as plastic lens, contact lens and artificial eye; optical materials such as optical filter, prism, mirror, and photographic material; stationery products such as tape and ink; display plates and devices and the surface-coating materials thereof, and the like. Alternatively, the polymer material according to the present invention may be used in cosmetic applications.

The shape (form) of the polymer material according to the present invention may be flat film, powder, spherical particle, crushed particle, bulky continuous particle, fiber, solenoid, hollow fiber, granule, plate, porous particle, or the other.

The polymer material according to the present invention, which contains the ultraviolet absorbent comprising the compound represented by formula (1) or (2), is superior in light resistance (ultraviolet fastness), causing no precipitation or bleed out of the ultraviolet absorbent during long-tem use. In addition, the polymer material according to the present invention, which has superior long-wavelength ultraviolet absorption capacity, can be used as an ultraviolet-absorbing filter or container, for protection, for example, of an ultraviolet-sensitive compound therein. It is possible to obtain a molded article (such as container) of the polymer material according to the present invention, for example, by molding the polymer substance by any molding method such as extrusion molding or injection molding. It is also possible to prepare a molded article coated with an ultraviolet-absorbing film made of the polymer material according to the present invention, by coating and drying a solution of the polymer substance on a separately prepared molded article.

When the polymer material according to the present invention is used as an ultraviolet-absorbing filter or film, the polymer substance is preferably transparent. Examples of the transparent polymer materials include polycarbonate, polyesters (e.g., polyethylene terephthalate, polyethylene naphthalate, polybutylene terephthalate, poly-1,4-cyclohexane dimethylene terephthalate, polyethylene 1,2-diphenoxyethane-4,4′-dicarboxylate, polybutylene terephthalate), and polymethyl methacrylate. Preferable are polycarbonate, polyethylene terephthalate, and acrylic resins. The polymer material according to the present invention may be used as a transparent support, and the transmittance of the transparent support in such a case is preferably 80% or more, more preferably 86% or more.

According to the present invention, it is possible to provide a polymer material that is superior in productivity when kneaded with a polymer or dissolved in a solvent, resistant to precipitation of the ultraviolet absorbent and bleeding out during long-term use, superior in long-wavelength ultraviolet absorption capacity, and superior in lightfastness while keeping the absorption capacity for an extended period of time.

The ultraviolet absorbent of the present invention is excellent in both a long-wavelength ultraviolet absorption capacity and light fastness, so that the ultraviolet absorbent is able to maintain the above-described absorption capacity for a long period of time. Further, the ultraviolet absorbent is also excellent in transparency, and when used in a polymer material, does not color the polymer material. In addition, the ultraviolet absorbent is also superior in convenience in handling, as it has a structure not irritant to the skin. Further, it is possible to incorporate the ultraviolet absorbent in a polymer material by kneading with a polymer substance, or dissolving in a solvent. Further, neither precipitation of the ultraviolet absorbent nor the bleed-out owing to a long-term use of the ultraviolet absorbent occurs in the produced polymer material.

The polymer material according to the present invention has advantageous effects that it is superior in production comformability when the ultraviolet absorbent is kneaded with a polymer or dissolved in a solvent, resistant to precipitation of the ultraviolet absorbent and bleeding out during long-term use, long-wavelength ultraviolet absorption capacity, and lightfastness (ultraviolet light fastness) while keeping the absorption capacity for an extended period of time. In addition, the polymer material containing the ultraviolet absorbent is also superior in convenience in handling, as it has a structure not irritant to the skin.

The polymer material according to the present invention, which has favorable lightfastness, can be used for polymeric molded products such as plastic, containers, coatings, coated films, fibers and construction materials. It can also be used, with its superior long-wavelength ultraviolet absorption capacity, in applications for protection of products sensitive to ultraviolet light, such as filter, packaging material, containers, coating, coated film, ink, fiber, construction material, recording medium, image display device and solar cell cover, and also in applications for prevention of decomposition of photo-sensitive compounds.

The polymer material according to the present invention can also be used in the cosmetic application. The cosmetic preparation containing the polymer material according to the present invention has advantageous effects that it is less in precipitation or yellowing of the ultraviolet absorbent during production of the cosmetic preparation, superior in long-wavelength ultraviolet absorption capacity and also in retention of the absorption capacity for an extended period of time. The cosmetic preparation containing the ultraviolet absorbent is also advantageous in that it has a structure not irritant to the skin.

In addition, the compound to be used in the polymer material according to the present invention has favorable effects that it has favorable long-wavelength ultraviolet absorption capacity, is resistant to precipitation or bleeding out when used in the polymer material and effective in improving lightfastness, as described above. Further, the compound can protect UV-sensitive organic materials, especially human and animal skins and hairs, from the damaging action by UV irradiation and is thus favorable as a photoprotecting agent for use in cosmetic products and pharmaceutical preparations for human and animals.

The present invention will be described in more detail based on the following examples. The materials, the amounts to be used, the proportions, the contents and procedures of treatment or processing, which will be shown in the examples, may be appropriately changed or modified, without departing from the spirit of the present invention. Therefore, the following examples are not interpreted as limiting of the scope of the present invention.

### EXAMPLES

### Example 1

### Preparation of Exemplified Compound (1)

The Exemplified compound (1) was prepared, in accordance with the following scheme:

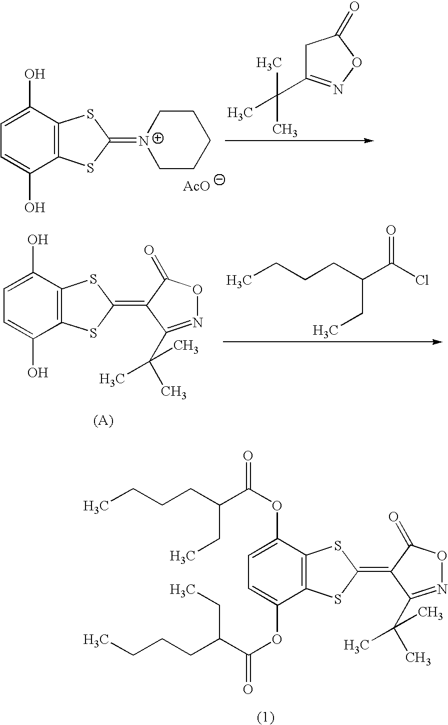

To 13.9 g (0.043 mol) of 1-(4,7-dihydroxybenzo[1,3]dithiol-2-ilydene)piperidinium acetate, 60 ml of N-methylpyrolidone and 6.0 g (0.043 mol) of 3-tert-butyl-5-isooxazolone were added and stirred at 80° C. for 4 hours under the nitrogen flow. Cooling to room temperature after reaction, the reaction liquid was added to 300 ml of a dilute hydrochloric acid (1N). Precipitated solid was separated by filtration. The thus-obtained solid was dispersed to 100 ml of acetonitrile and stirred at 70° C. for 30 minutes and then allowed to cool to room temperature. The thus-obtained crystals were separated by filtration, thereby to obtain 11.5 g of intermediate (A) (yield 84%). Then, 10.0 g (0.031 mol) of the intermediate (A) thus obtained was dissolved in 100 ml of N,N-dimethylacetamide and then 10.4 ml (0.075 mol) of triethylamine was added thereto and cooled to 0° C. Thereafter, 11.8 ml (0.068 mol) of 2-ethylhexanoyl chloride was added dropwise thereto, and the temperature of the reaction liquid was returned to room temperature and then stirred for 4 hours. To the reaction liquid, 500 ml of ethyl acetate and 500 ml of a dilute hydrochloric acid (1 N) were each added and then liquid separation was conducted using a separating funnel. The thus-separated organic layer was washed with a 3% sodium hydrogen carbonate aqueous solution and a saturated saline. After dehydration with magnesium sulfate, the magnesium sulfate was separated by filtration. A solvent of the organic layer was distilled away under reduced pressure. The thus-obtained solid was purified by silica gel column chromatography (hexane/ethyl acetate=10/1) to thereby obtain the Exemplified compound (1). Yield: 13.0 g, Yield Percentage: 73%

¹H NMR (CDCl₃) δ 7.25 (2H), 2.62 (2H), 1.60-1.90 (8H), 1.51 (9H), 1.35-1.5 (8H), 1.10 (6H), 0.95 (6H) ppm

### Example 2

### Preparation of Exemplified Compound (16)

The Exemplified compound (16) was prepared, in accordance with the following scheme:

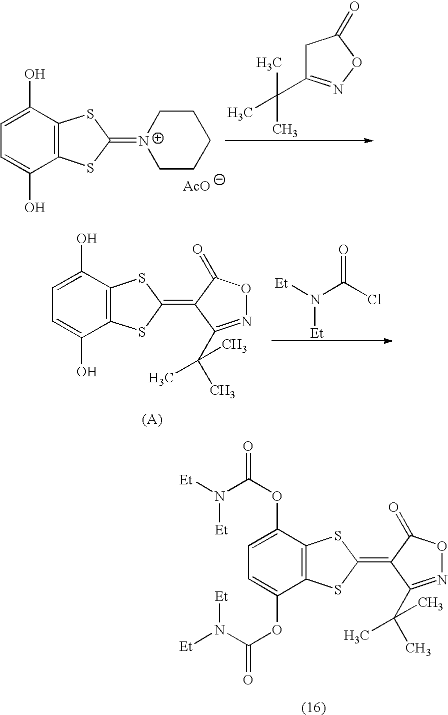

The intermediate (A) in an amount of 0.5 g (0.0015 mol) obtained in the same manner as in the method in Example 1, was dissolved in 10 ml of N,N-dimethylacetamide, and then, thereto, 0.53 g (0.0038 mol) of potassium carbonate and 0.43 ml (0.0034 mol) of N,N-diethylcarbamic acid chloride were added, followed by stirring at room temperature for 5 hours under the nitrogen flow. To the reaction liquid, 50 ml of ethyl acetate was added, and insoluble matters were separated by filtration. To the filtrate, 50 ml of dilute hydrochloric acid (1 N) was added and then a liquid separation was conducted using a separating funnel. Then, the separated organic layer was washed with a 3% sodium hydrogen carbonate aqueous solution and a saturated saline. After dehydration with magnesium sulfate, the magnesium sulfate was separated by filtration. A solvent of the organic layer was distilled away under reduced pressure. The thus-obtained solid was purified by silica gel column chromatography (hexane/ethyl acetate=3/1) to thereby obtain the Exemplified compound (16). Yield: 0.5 g, Yield Percentage: 62%

¹H NMR (CDCl₃) δ 7.37 (2H), 3.35-3.60 (8H), 1.52 (9H), 1.35 (6H), 1.23 (6H) ppm

### Example 3

### Preparation of Exemplified Compound (45)

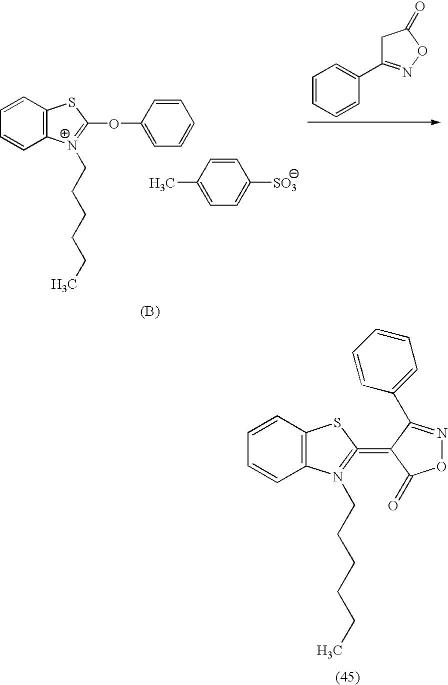

In 10 ml of acetonitrile, 2.1 g (0.0043 mol) of the compound (B) and 0.7 g (0.0043 mol) of 3-phenyl-5-isooxazolone were dissolved and then 0.6 ml (0.0043 mol) of triethylamine was added, and stirred for 3 hours while heating at 50° C. Cooling the reaction liquid to room temperature, 50 ml of ethyl acetate and 50 ml of water were each added thereto and then liquid separation was conducted using a separating funnel. The organic layer was dehydrated with magnesium sulfate, the magnesium sulfate was separated by filtration. A solvent of the organic layer was distilled away under reduced pressure. The thus-obtained solid was purified by silica gel column chromatography (hexane/ethyl acetate=1/1) to thereby obtain the Exemplified compound (45). Yield: 0.3 g, Yield Percentage: 18%

¹H NMR (CDCl₃) δ 7.38-7.65 (9H), 4.65 (2H), 1.75 (2H), 1.23 (6H), 0.85 (3H) ppm

Further, other exemplified compounds can be synthesized by referring to the above-described synthetic methods.

### (Evaluation)

### <Evaluation of Absorption Properties>

Regarding Examplified compounds (1), (16) and (45) as well as compounds (1) and (2) for comparison, a solution in ethyl acetate of the respective compound at a concentration of approximately 10×10⁻⁶ mol·dm⁻³ was prepared, and the UV spectrum of the solution was measured by a spectrophotometer UV-3600 (trade name) manufactured by Shimadzu Corporation using it in a 1-cm quartz cell. The absorption maximum wavelength, the molar extinction coefficient at the absorption maximum wavelength, and the absorbances at 400 nm and 420 nm with respect to the absorbance at the absorption maximum wavelength, were calculated form the spectral chart obtained. The results are shown in Table 3 below.

TABLE 3

| | | | Ratio (%) of | Ration (%) of | |
|---|---|---|---|---|---|
| | | | absorbance at | absorbance at | |
| | | | 400 nm to | 420 nm to | |
| | | Molar extinction | absorbance at | absorbance at | |
| | Absorption | coefficient at | absorption | absorption | |
| | maximum | absorption maximum | maximum | maximum | |
| Compound | wavelength (nm) | wavelength | wavelength | wavelength | Remarks |
| Exemplified compound (1) | 384 | 25,000 | 77 | 8 | This invention |
| Exemplified compound (16) | 385 | 26,000 | 83 | 11 | This invention |
| Exemplified compound (45) | 383 | 24,000 | 65 | 15 | This invention |
| Comparative compound (1) | 348 | 61,000 | 0.4 | 0 | Comparative example |
| Comparative compound (2) | 374 | 19,000 | 61 | 17 | Comparative example |

Comparative
compound (1)

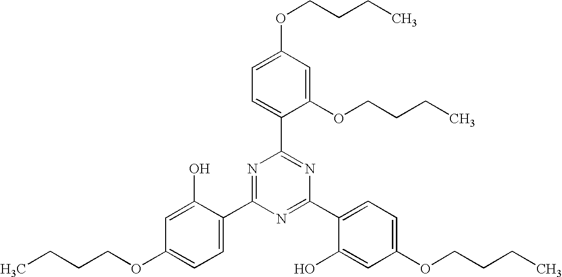

Comparative
compound (2)

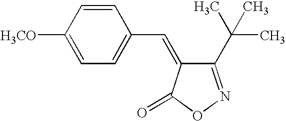

As is apparent from the results shown in Table 3, the ultraviolet absorbent of the present invention containing the compound represented by formula (1) or (2) has a sufficient absorbance in an ultraviolet range up to 400 nm, whereby it is expected that ultraviolet rays around this wavelength are effectively blocked. Further, since the molar extinction coefficient of the ultraviolet absorbent of the present invention at the wavelength of maximum absorption is more than 20,000, it is expected that a sufficient shielding ability of the ultraviolet ray is obtained in a small usage. In contrast, since the comparative compound (1) has almost no absorption around 400 nm, the shielding ability of the ultraviolet ray around this wavelength cannot be expected. Further, even though the comparative compound (2) has a sufficient absorption at the wavelength of 400 nm, much of the absorption still remains even at 420 nm, which causes coloring. Contrary to the above, the ultraviolet absorbent of the present invention has such a characteristic waveform that absorption at long-wavelength side is sharp. Therefore, the absorbance of these absorbents at 420 nm is sufficiently-small. Even by comparison with the naked eye, there was a clear difference in coloring between the ultraviolet absorbents of the present invention and the Comparative compound (2).

### Example 4

### Preparation of Kneaded Ultraviolet Absorbent-Containing Polymer Film

The exemplified compound (16) was added to 5 g polyethylene terephthalate in the preparation of a 50 μm film so as to be an absorbance of 1.0 at the absorption maximum wavelength, and the mixture was melt-kneaded at 265° C. and cooled, to give a bulk of ultraviolet absorbent-containing polyethylene terephthalate. The ultraviolet absorbent-containing polyethylene terephthalate was stretched at 280° C., to give an ultraviolet absorbent-containing polymer film sample.

The UV spectrum of the thus-produced film was measured using a spectrophotometer UV-3600 (trade name, manufactured by Shimadzu). As a result, a spectrum was obtained almost equal to the spectrum obtained from the above-described ethyl acetate solution.

### Example 5

### Preparation of Polymer Film Samples 101 to 105

In 5.0 g of a 10.4% dichloromethane solution of DIANAL BR80 (trade name, manufactured by MITSUBISHI RAYON CO., LTD, acrylic resin, Tg=105° C.), 0.020 g of the Exemplified compound (1) was dissolved. The resultant solution was applied with a wire bar (hereinafter, referred to as a bar coat) on a slide glass so that the dry film thickness would be about 5 μm and the absorbance of the polymer film at the maximum absorption wavelength would be about 1. The thus-obtained coating was dried to thereby produce a polymer film sample 101 having an ultraviolet absorbing ability.

A polymer film sample 102 was produced in the same manner as the polymer film sample 101, except that the Exemplified compound (1) was changed to the Exemplified compound (16) and the addition amount of the compound was adjusted so that the absorbance of the polymer film at the maximum absorption wavelength would be about 1.

Similarly, polymer film samples 103 and 104 were produced using the comparative compound (1) and the comparative compound (2) respectively in place of the Exemplified compound (1) in the same manner as the polymer film sample 101.

The UV spectrum of each of the thus-produced films was measured using a spectrophotometer UV-3600 (trade name, manufactured by Shimadzu). As a result, each spectrum was obtained almost equal to the spectrum obtained from the above-described ethyl acetate solution.

### (Evaluation)

### <Compulsory Evaluation of Light Fastness>

The absorbance of each polymer film was measured using a spectrophotometer UV-3600 (trade name, manufactured by Shimadzu), to a reference of a polymer film free of the ultraviolet absorbent. Xenon light (170,000 lux) was irradiated to each polymer film for 24 hours, and a residual ratio after irradiation of the ultraviolet absorbent used in the each polymer film was measured. The residual ratio was calculated according to the following formula:

Residual ratio (%)=100×(Absorbance after irradiation)/(Absorbance before irradiation).

Note that the absorbance is a value obtained by measurement at the absorption maximum wavelength (λmax) of the respective polymer film.

The results are shown in Table 4.

TABLE 4

| Polymer | Ultraviolet absorbent | Residual | |
|---|---|---|---|
| film No. | compound | ratio (%) | Remarks |
| 101 | Exemplified compound (1) | 93 | This invention |
| 102 | Exemplified compound (16) | 88 | This invention |
| 103 | Comparative compound (1) | 99 | Comparative |
| | | | example |
| 104 | Comparative compound (2) | 69 | Comparative |
| | | | example |

As is apparent from the results in Table 4, light fastness of each polymer film according to the present invention is close to the level of the comparative compound (1) that is a general-purpose ultraviolet absorbent.

### Example 6

### Production of 410 nm Cut Filter Samples 201 to 204

Any one of the Exemplified compounds (1) and (16) and the comparative compounds (1) and (2) were respectively dissolved in 2.54 g of a 29.1% solution (toluene/methyl ethyl ketone 1:1 mixed solvent) of VYLON 200 (trade name, manufactured by TOYOBO CO., LTD, Tg=67° C.) in quantities by weight of the UV absorbent shown in the below-described Table 5 that are calculated so that transmittance of the UV absorbent at the wavelength of 410 nm would be 1% or less. The resultant solution was applied by a bar coat in the same manner as in Example 5 so that the dry film thickness would be about 25 μm, to thereby produce 410 nm cut filter samples 201 to 204.

### (Evaluation)

Evaluation with the naked eye was performed, with respect to coloring and bleed-out of the thus-produced cut filters. The degree of the coloring was evaluated on the following criteria for assessment.

### <Criteria of Coloring>

A: Yellow coloring is a little.
B: Coloring is visible to some extent.
C: Coloring is conspicuous.

Further, the degree of the bleed-out was evaluated on the following criteria for assessment.

### <Criteria of Bleed-Out>

A: There is no appearance of bleed-out.
B: There is some appearance of bleed-out.
C: There is conspicuous appearance of bleed-out

The results are shown in Table 5.

TABLE 5

| Cut | Ultraviolet | Addition | | | |
|---|---|---|---|---|---|
| filter | absorbent | amount | | Bleed- | |
| No. | compound | (g) | Coloring | out | Remarks |
| 201 | Exemplified | 0.0252 | A | A | This |
| | compound (1) | | | | invention |
| 202 | Exemplified | 0.0127 | A | A | This |
| | compound (16) | | | | invention |
| 203 | Comparative | 2.2 | C | C | Comparative |
| | compound (1) | | | | example |
| 204 | Comparative | 0.0107 | C | A | Comparative |
| | compound (2) | | | | example |

As is apparent from the results shown in the Table 5, objected filters were produced by using the Exemplified compounds (1) or (16) in definitely less amount than that of the comparative compound. Specifically, when the comparative compound (1) is used, a desirable transmittance was not obtained unless the UV absorbent was added in large quantity. As a result, there was conspicuous appearance of bleed-out in the filter sample 203 and a uniform film was not produced. Further, a yellow coloring was conspicuous in the filter sample 204, which was completely unsuitable for the optical transparency-requiring use.

In contrast, coloring of the filter samples 201 and 202 was each much less than comparative compounds, although a little coloring of the filter is inevitable from the nature of 410 nm cut. Further, uniform filters with no occurrence of bleed-out can be produced regarding the filter samples 201 and 202.

The results of evaluations of each polymer film in the above Examples in terms of long wave-blocking ability and light fastness are shown together in Table 6. The long wave-blocking ability was determined by a ratio of absorbance at 400 nm relative to absorbance at the absorption maximum wavelength according to the following three grade criteria for assessment:

### <Criteria of Long Wave-Blocking Ability>

A: Ratio of absorbance at 400 nm relative to absorbance at the absorption maximum wavelength is 70% or more
B: Ratio of absorbance at 400 nm relative to absorbance at the absorption maximum wavelength is 30% or more, but less than 70%
C: Ratio of absorbance at 400 nm relative to absorbance at the absorption maximum wavelength is less than 30%

Coloring was determined by observation with the naked eye of the polymer film produced in Example 5 according to the following three grade criteria for assessment:

### <Criteria of Coloring>

A: No coloring is observed.
B: Some coloring is observed.
C: Apparent yellow coloring is observed.

Light fastness was determined by a residual ratio after irradiation to xenon light for 24 hours according to the following three grade criteria for assessment:

### <Criteria of Light Fastness>

A: Residual ratio is 80% or more
B: Residual ratio is 50% or more, but less than 80%
C: Residual ratio is less than 50%

TABLE 6

| | | Long | | | |
|---|---|---|---|---|---|
| Polymer | Ultraviolet | wave- | | | |
| film | absorbent | blocking | | Light | |
| No. | compound | ability | Coloring | fastness | Remarks |
| 101 | Exemplified | A | B | A | This |
| | compound (1) | | | | invention |
| 102 | Exemplified | A | B | A | This |
| | compound (16) | | | | invention |
| 103 | Comparative | C | A | A | Comparative |
| | compound (1) | | | | example |
| 104 | Comparative | B | C | B | Comparative |
| | compound (2) | | | | example |

As is apparent from the results in Table 6, the polymer film samples 101 to 102 according to the present invention can effectively block ultraviolet rays of long-wavelength, particularly round 400 nm that is difficult to be effectively blocked by hitherto known ultraviolet absorbents. Further, it is understood that the polymer film samples 101 to 102 according to the present invention each are excellent in light fastness.

Having described our invention as related to the present embodiments, it is our intention that the invention not be limited by any of the details of the description, unless otherwise specified, but rather be construed broadly within its spirit and scope as set out in the accompanying claims.

This non-provisional application claims priority under 35 U.S.C. §119(a) on Patent Application No. 2008-276103 filed in Japan on Oct. 27, 2008, which is entirely herein incorporated by reference.

What we claim is:

1. An ultraviolet absorbent, comprising a compound represented by formula (1): 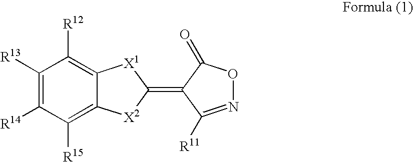
wherein X¹ and X² each independently represent an oxygen atom, a sulfur atom, or —NR¹⁶—; R¹¹, R¹², R¹³, R¹⁴, R¹⁵ and R¹⁶ each independently represent a hydrogen atom or a monovalent substituent.

2. The ultraviolet absorbent according to claim 1, wherein, in formula (1), X¹ and X² each are a sulfur atom.

3. A polymer material, comprising:
an ultraviolet absorbent which comprises a compound represented by formula (1); and
at least one kind of a polymer substance: 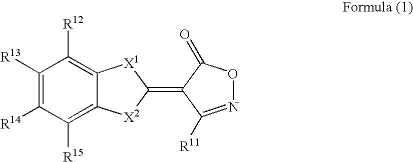
wherein X¹ and X² each independently represent an oxygen atom, a sulfur atom, or —NR¹⁶—; R¹¹, R¹², R¹³, R¹⁴, R¹⁵ and R¹⁶ each independently represent a hydrogen atom or a monovalent substituent.

4. The polymer material according to claim 3, wherein, in formula (1), X¹ and X² each are a sulfur atom.

5. The polymer material according to claim 3, wherein the polymer substance is at least one selected from the group consisting of acrylic acid-based polymers, polyester-based polymers and polycarbonate-based polymers.

6. The polymer material according to claim 3, wherein a glass transition point (Tg) of the polymer substance is −80° C. or higher and 200° C. or lower.

7. The polymer material according to claim 3, wherein the polymer substance is a polyacrylate ester, a polycarbonate or a polyethylene terephthalate.

8. The polymer material according to claim 3, wherein the polymer substance is the polyethylene terephthalate; and wherein the ultraviolet absorbent is contained in an amount of 0.1 mass % to 50 mass % with respect to 100 mass % of the polyethylene terephthalate.

9. The polymer material according to claim 8, wherein the polymer material is prepared by melt-kneading of the polyethylene terephthalate and the ultraviolet absorbent at a temperature of 200° C. or higher.

10. The polymer material according to claim 3, wherein the polymer substance is the polyacrylate ester or the polycarbonate; and wherein the ultraviolet absorbent is contained in an amount of 0.1 mass % to 50 mass % with respect to 100 mass % of the polyacrylate ester or polycarbonate.

11. The polymer material according to claim 10, wherein the polymer material is prepared by dissolving the polyacrylate ester or the polycarbonate and the ultraviolet absorbent in a solvent having a boiling point of 200° C. or lower to give a solution, and applying the solution on a base substrate.

12. A compound represented by formula (2): 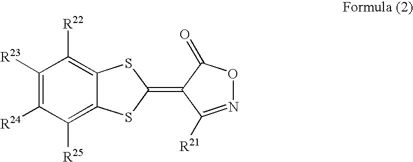
wherein R²¹ represents a substituted or unsubstituted alkyl group; and R²², R²³, R²⁴ and R²⁵ each independently represent a hydrogen atom or a monovalent substituent.
